(19) United States
(12) Patent Application Publication
(10) Pub. No.: US 2020/0408762 A1
(43) Pub. Date: Dec. 31, 2020

(54) SYSTEMS AND METHODS OF DIAGNOSING AND PROGNOSING CANCER

(71) Applicants: DIGNITY HEALTH, Phoenix, AZ (US); THE TRANSLATIONAL GENOMICS RESEARCH INSTITUTE, Phoenix, AZ (US)

(72) Inventors: Landon Inge, Phoenix, AZ (US); Timothy Whitsett, Phoenix, AZ (US); Patrick Pirrotte, Phoenix, AZ (US); Ross Bremner, Phoenix, AZ (US); Khyatiben Pathak, Phoenix, AZ (US)

(21) Appl. No.: 16/978,638

(22) PCT Filed: Mar. 7, 2019
(86) PCT No.: PCT/US2019/021216

§ 371 Date: Sep. 4, 2020

Related U.S. Application Data
(60) Provisional application No. 62/639,546, filed on Mar. 7, 2018.

(51) Int. Cl. G01N 33/574

(52) U.S. Cl. CPC G01N 33/57407; G01N 2405/08; G01N 2405/04

(57) ABSTRACT

The present invention provides a method for monitoring esophageal adenocarcinoma (EAC) disease progression in a subject, the method comprising: obtaining a biological sample from the subject; measuring with a quantitative analytical method at least one metabolite; determining a metabolomic biosignature of EAC disease progression based on a comparison of quantitative data for the at least one metabolite to corresponding data obtained for at least one reference sample; and identifying active EAC disease progression in the subject if the quantity of the at least one metabolite in the sample from the subject is greater than that found in the at least one reference sample.

### CROSS REFERENCE TO RELATED APPLICATIONS

This application claims priority to and the benefit of U.S. Provisional Application Ser. No. 62/639,546, filed on Mar. 7, 2018, the contents of which are hereby incorporated by reference in their entirety.

### TECHNICAL FIELD

The present invention relates to the field of diagnosis and prognosis of a common type of esophageal cancer known as esophageal adenocarcinoma.

### BACKGROUND

The esophageal cancer is an aggressive form of cancer with 5-year survival rate of 19% (1). The American Cancer Society estimated nearly 17,650 new cases and 16,080 deaths related to esophageal cancer in 2019 (2). The esophageal adenocarcinoma (EAC), a cancer of mucus-secreting glands is the most common type of esophageal cancer in the United States, affecting primarily white men (3). The long term gastroesophageal reflux disease (GERD), Barrett's esophagus (BE), smoking and obesity are common risk factors contributing to pathological progression (4). Chronic inflammation caused by gastric reflux is the major background for EAC development which undergoes metaplastic change of an intestinalized epithelium called BE (3). This metaplasia further progresses into dysplasia and EAC. Long term disease monitoring in at risk population, such as BE and GERD patients, may improve overall survival rate. However, only a small percentage of these populations progress into an advanced stage (5-7). The existing disease management practice is heavily dependent upon imaging based diagnosis and chemoradiotherapy (8, 9). The imaging based diagnosis involves esophago-gastro duodenoscopy, CT scans and biopsy, which are invasive, expensive and inadequate for early detection (8-10). There is an unmet need for molecular signatures that accurately classify disease stages and predict early progression (8, 11). Identification of a minimally invasive and sensitive assay to provide disease stratification and early detection of EAC would significantly improve the current standard of care in EAC patients and decrease the mortality rate.

Tumor cells are known to undergo constant molecular reprogramming to meet their energy demand in order to promote cell proliferation and survival (12-14). As a result of this, metabolite levels and pathways are significantly deregulated in many cancers (15-18). Metabolomics has been explored as a promising approach to capture metabolic changes such as the Warburg effect, amino acid metabolism, lipid turnover, and oxidative stress in cancers (19-22). There is a need to develop more efficient methods of monitoring EAC disease progression to facilitate early detection of the disease and provide effective therapeutic treatments.

### SUMMARY

Previously, metabolomics studies utilized LC-MS/MS based discovery and targeted approaches in serum to determine molecular alterations associated with EAC (23, 24). However, metabolites in serum are largely associated with systemic changes and are not as specific as metabolites collected at the site of origin. Sampling tissues in this context may provide localized snapshots of metabolic alterations but invasiveness of this approach hinders its further exploration in fore gut cancers. In addition, the majority of the studies with tissue have focused on squamous cell carcinoma rather than adenocarcinoma (25-27).

In reflux disease, the lower esophagus is exposed to gastric fluid which may cause esophageal injury. Animal studies have shown reflux of gastric fluid into esophagus is the main risk factor of GERD and can cause severe esophageal injury, metaplasia and development of EAC (28, 29). Gastric fluid may serve as a surrogate of esophageal microenvironment to study EAC. However, gastric fluid has been minimally explored towards understanding EAC progression in human. Signatures of metabolic reprogramming linked to EAC may be preserved in gastric fluid which can stratify stages of disease. In the present invention, targeted metabolomics was employed on cell models of metaplasia, dysplasia and EAC to determine metabolic alterations associated with disease progression. These observations were further verified in gastric fluids of GERD, metaplasia, dysplasia and EAC patients.

The present invention provides a method for monitoring EAC disease progression in a subject, the method comprising: obtaining a biological sample from the subject; measuring with a quantitative analytical method at least one metabolite selected from citrulline, C0, SM OH C16:1, glutamic acid, creatinine, histidine, glycine, phenylalanine, methionine, glutamine, serine, tyrosine, alanine, isoleucine, SM C24:0, C2, SM C16:0, taurine, valine, SM OH C22:1, leucine, sarcosine, SM C24:1, threonine, asparagine, lysine, Met-SO, SM C18:0, proline, t4-OH-Pro, C3-DC C4-OH, SM C26:0, aspartic acid, C14:2-OH, histamine, and combinations thereof; determining a metabolomic biosignature of EAC disease progression based on a comparison of quantitative data for the at least one metabolite to corresponding data obtained for at least one reference sample; and identifying active EAC disease progression in the subject if the quantity of the at least one metabolite in the sample from the subject is greater than that found in the at least one reference sample.

In another aspect, the present invention provides a method for detecting EAC in a subject, the method comprising: obtaining a biological sample from the subject; measuring with a quantitative analytical method at least one metabolite selected from citrulline, C0, SM OH C16:1, glutamic acid, creatinine, histidine, glycine, phenylalanine, methionine, glutamine, serine, tyrosine, alanine, isoleucine, SM C24:0, C2, SM C16:0, taurine, valine, SM OH C22:1, leucine, sarcosine, SM C24:1, threonine, asparagine, lysine, Met-SO, SM C18:0, proline, t4-OH-Pro, C3-DC C4-OH, SM C26:0, aspartic acid, C14:2-OH, histamine, and combinations thereof; determining a metabolomic biosignature of EAC disease progression based on a comparison of quantitative data for the at least one metabolite to corresponding data obtained for at least one reference sample; and detecting EAC in the subject if the quantity of the at least one metabolite in the sample from the subject is greater than that found in the at least one reference sample.

In certain aspects, the at least one metabolite comprises a panel of metabolites selected from the group consisting of: a) a panel comprising taurine, methionine, methionine sulfoxide (Met-SO), and trans-4-hydroxyproline (t4-OH-Pro); b) a panel comprising glutamic acid, glycine, histidine, phenylalanine, methionine, serine, glutamine, tyrosine, alanine, isoleucine, valine, leucine, threonine, asparagine, lysine, proline, and aspartic acid; and c) a panel comprising citrulline, C0, SM OH C16:1, glutamic acid, creatinine, histidine, glycine, phenylalanine, methionine, glutamine, serine, tyrosine, alanine, isoleucine, SM C24:0, C2, SM C16:0, taurine, valine, SM OH C22:1, leucine, sarcosine, SM C24:1, threonine, asparagine, lysine, Met-SO, SM C18:0, proline, t4-OH-Pro, C3-DC C4-OH, SM C26:0, aspartic acid, C14:2-OH, and histamine.

In one embodiment, the panel of metabolites comprises glutamic acid, glycine, histidine, phenylalanine, methionine, serine, glutamine, tyrosine, alanine, isoleucine, valine, leucine, threonine, asparagine, lysine, proline, and aspartic acid.

In other embodiments, the method further comprises measuring with a quantitative analytical method at least one additional metabolite selected from tryptophan, C4, C14:1-OH, SM (OH) C24:1, spermidine, SM (OH) C22:2, SM C26:1, C16-OH, C10:2, putrescine, spermine, C14:1, C8, C5, PC aa C42:1, and combinations thereof to determine the metabolomic biosignature of EAC disease progression.

In certain embodiments, the quantity of the at least one metabolite in the sample from the subject is at least 2.0 times greater than that found in the at least one reference sample.

In some aspects, the quantitative analytical method comprises mass spectrometry. In on aspect, the mass spectrometry comprises liquid chromatography-tandem mass spectrometry (LC-MS/MS). The LC-MS/MS may further comprise analyzing in multiple reaction monitoring (MRM) in positive mode of electrospray ionization (ESI).

In one embodiment, the at least one reference sample is obtained from at least one reference subject with metaplasia. In another embodiment, the at least one reference sample is obtained from at least one reference subject with dysplasia. In one aspect, the biological sample from the subject and the at least one reference sample are gastric fluid samples.

In certain embodiments, active EAC disease progression is progression of the disease from metaplasia to dysplasia. In other embodiments, active EAC disease progression is progression of the disease from dysplasia to EAC.

In yet other embodiments, the present invention relates to a method for monitoring EAC disease progression in a subject, the method comprising: obtaining a biological sample from the subject; measuring with a quantitative analytical method at least one metabolite selected from Met-SO, Met-SO/Met, asymmetric dimethyl arginine (ADMA), t4-OH-Pro, alanine, isoleucine, tyrosine, and combinations thereof; determining a metabolomic biosignature of EAC disease progression based on a comparison of quantitative data for the at least one metabolite to corresponding data obtained for at least one reference sample; and identifying active EAC disease progression in the subject if the quantity of the at least one metabolite in the sample from the subject is greater than that found in the at least one reference sample. As used herein, “Met-SO/Met” refers to the ratio of methionine sulfoxide to methionine quantified in a sample.

In one aspect, the present invention provides a method for detecting EAC in a subject, the method comprising: obtaining a biological sample from the subject; measuring with a quantitative analytical method at least one metabolite selected from Met-SO, Met-SO/Met, asymmetric dimethyl arginine (ADMA), t4-OH-Pro, alanine, isoleucine, tyrosine, and combinations thereof; determining a metabolomic biosignature of EAC disease progression based on a comparison of quantitative data for the at least one metabolite to corresponding data obtained for at least one reference sample; and detecting EAC in the subject if the quantity of the at least one metabolite in the sample from the subject is greater than that found in the at least one reference sample.

In some embodiments, the quantity of the at least one metabolite in the sample from the subject is at least 1.5 times greater than that found in the at least one reference sample.

In some aspects, the present invention relates to a method for monitoring EAC disease progression in a subject, the method comprising: obtaining a biological sample from the subject; measuring with a quantitative analytical method at least one metabolite selected from arginine, proline, serine, glutamine, histidine, a glycerophospholipid, a sphingolipid, and combinations thereof; determining a metabolomic biosignature of EAC disease progression based on a comparison of quantitative data for the at least one metabolite to corresponding data obtained for at least one reference sample; and identifying active EAC disease progression in the subject if the quantity of the at least one metabolite in the sample from the subject is less than that found in the at least one reference sample.

In other aspects, the present invention relates to a method for detecting EAC in a subject, the method comprising: obtaining a biological sample from the subject; measuring with a quantitative analytical method at least one metabolite selected from arginine, proline, serine, glutamine, histidine, a glycerophospholipid, a sphingolipid, and combinations thereof; determining a metabolomic biosignature of EAC disease progression based on a comparison of quantitative data for the at least one metabolite to corresponding data obtained for at least one reference sample; and detecting EAC in the subject if the quantity of the at least one metabolite in the sample from the subject is less than that found in the at least one reference sample.

In one embodiment, the glycerophospholipid is lysoPC a C26:0, PC aa C28:1, PC aa C34:2, PC aa C36:1, PC aa C36:2, PC aa C40:4, PC aa C42:1, PC ae C36:2, PC ae C38:1, PC ae C42:2, PC ae C42:3, or a combination thereof. In another embodiment, the sphingolipid is SM C20:2, SM OH C16:1, SM OH C22:1, or a combination thereof.

In certain aspects, the quantity of the at least one metabolite in the sample from the subject is about 1.5 times less than that found in the at least one reference sample.

### BRIEF DESCRIPTION OF THE DRAWINGS

FIG. 1 depicts the experimental outline for analyzing cell lines modeling Metaplasia, Dysplasia and esophageal adenocarcinoma and gastric fluid with matched plasma samples from different patient populations.

FIG. 2 depicts a quantitative metabolomics workflow for analyzing metabolites in Metaplasia, Dysplasia and esophageal adenocarcinoma groups.

FIG. 3A depicts hierarchical clustering of Metaplasia, Dysplasia and esophageal adenocarcinoma groups for intracellular metabolites. FIG. 3B depicts hierarchical clustering of Metaplasia, Dysplasia and esophageal adenocarcinoma groups for extracellular metabolites.

FIG. 4 depicts altered metabolic pathways in esophageal adenocarcinoma progression.

FIG. 5 depicts metabolic changes in esophageal adenocarcinoma progression. Abbreviations: GERD: gastroesophageal reflux disease; Dys: dysplasia; Met: metaplasia; EA: esophageal carcinoma.

FIG. 6 depicts a univariate and multivariate logistic regression and receiver operating curve with 21 metabolites identified in a metabolomics analysis.

FIGS. 7A, 7B, and 7C depict an overview of the study cohort and workflow. FIG. 7A depicts the intracellular and extracellular metabolism investigated using metaplasia (CPA), high grade dysplasia (CPB, CPC, CPD) and EAC (SK-GT, OE19, OE33) cell lines derived from patients. FIG. 7B depicts the gastric fluid metabolomics studied in a cross-sectional patient cohort of GERD, metaplasia, dysplasia and EAC patients. FIG. 7C depicts a quantitative metabolomics workflow comprised of phenyl isothiocynate (PITC) derivatization of amino acids followed by metabolite extraction from cell lysate, culture media, and gastric fluid and quantitative analysis using isotopically labeled internal standards. Metabolites were validated with MetIDQ software against human plasma and subjected to further downstream data analysis and pathway analysis.

FIGS. 8A and 8B depict the dynamics of intracellular and extracellular metabolism in dysplasia, metaplasia and EAC cell models. FIG. 8A depicts hierarchical clustering showing relative abundance of metabolites that are significantly different between three disease models (adj. p<0.05) at intracellular (left panel) and extracellular (right panel) levels. The key indicates normalized concentration of metabolites. Disease groups are metaplasia, dysplasia, and EAC. FIG. 8B depicts the distribution of significant metabolites in their respective metabolic class in both intra- (left) and extracellular (right) medium.

FIG. 9 depicts a schematic of deregulated metabolic pathways in dysplasia to metaplasia (left arrows in each box) and EAC to metaplasia (right arrows in each box) in both intracellular and extracellular metabolism. EAC or EA refers to esophageal adenocarcinoma.

FIGS. 10A, 10B, and 10C depict metabolic changes in gastric fluid of metaplasia and dysplasia patients. FIG. 10A presents a volcanoplot showing difference in metabolite concentration from metaplasia to dysplasia. Significant metabolites (above the dashed line, >−log 10(adj p)1.3013) are down-regulated in metaplasia. FIG. 10B depicts a principal component analysis using significant metabolites of metaplasia and dysplasia patients showing heterogeneity in the disease groups. FIG. 10C depicts hierarchical clustering using significant metabolites of metaplasia and dysplasia patients and indicates heterogeneity in the disease group.

FIG. 11A depicts box plots showing gastric concentration of representative significant metabolites (Adj. p<0.05) in metaplasia and dysplasia. Concentrations were measured in units of μM. FIG. 11B depicts significant enrichment in metabolic pathways in gastric fluid of dysplasia patients. FIG. 11C depicts significant enrichment in metabolic enzymes in gastric fluid of dysplasia patients.

FIG. 12 depicts a receiver operative curve analysis of three models with AUC>0.9. Model 1 is a combined panel of 17 significant amino acids, model 2 is a panel of all 35 significant metabolites, and panel 3 is comprised of taurine, Met, Met-SO, and t4-OH-Pro.

FIG. 13 depicts a schematic representation of modulation of the gastric fluid metabolome in metaplasia and dysplasia patients.

### DETAILED DESCRIPTION

As used herein, the verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”.

Metabolites may be detected using any suitable quantitative analytical method including, but not limited to, liquid and gas phase chromatography, alone or coupled to mass spectrometry (See e.g., experimental section below), NMR (See e.g., US patent publication 20070055456, herein incorporated by reference), immunoassays, chemical assays, spectroscopy and the like. In some embodiments, commercial systems for chromatography and/or NMR analysis are utilized.

In other embodiments, metabolites are detected using optical imaging techniques such as magnetic resonance spectroscopy (MRS), magnetic resonance imaging (MRI), CAT scans, ultra sound, MS-based tissue imaging or X-ray detection methods (e.g., energy dispersive x-ray fluorescence detection).

Any suitable quantitative analytical method may be used to analyze the sample in order to determine the presence, absence or level(s) of the one or more metabolites in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, biochemical or enzymatic reactions or assays, and combinations thereof. Further, the level(s) of the one or more metabolites may be measured indirectly, for example, by using an assay that measures the level of a compound (or compounds) that correlates with the level of the metabolite(s) that are desired to be measured.

The levels of one or more of the recited metabolites may be determined in the methods of the present invention. For example, the level(s) of one metabolites, two or more metabolites, three or more metabolites, four or more metabolites, five or more metabolites, six or more metabolites, seven or more metabolites, eight or more metabolites, nine or more metabolites, ten or more metabolites, etc., including a combination of some or all of the disclosed metabolites may be determined and used in such methods.

Determining levels of combinations of the metabolites may allow greater sensitivity and specificity in the methods, such as diagnosing esophageal cancer and aiding in the diagnosis of esophageal cancer, and may allow better differentiation or characterization of esophageal cancer from other disorders or other cancers that may have similar or overlapping metabolites to esophageal cancer (as compared to a subject not having esophageal cancer). For example, ratios of the levels of certain metabolites in biological samples may allow greater sensitivity and specificity in diagnosing esophageal cancer and aiding in the diagnosis of esophageal cancer and allow better differentiation or characterization of esophageal cancer from other cancers or other disorders of the esophagus and digestive system that may have similar or overlapping metabolites to esophageal cancer (as compared to a subject not having esophageal cancer).

In certain aspects, mass spectrometry is used to determine relative levels of metabolites between samples and control or reference samples. Liquid chromatography-mass spectrometry (LC-MS) combines the physical separation of liquid chromatography (including high performance liquid chromatography) with the mass analysis of mass spectrometry (MS). LC-MS has high sensitivity and selectivity and is useful for separation, detection and identification of chemicals.

In other aspects, “targeted”, quantitative metabolomics is performed with ¹H nuclear magnetic resonance (NMR) spectroscopy as outlined in Pan et al., “Comparing and combining NMR spectroscopy and mass spectrometry in metabolomics” Anal Bioanal Chem 2007; 387(2):525-527.

Some embodiments of the invention may include a method of comparing a metabolite in a sample relative to one or more control or reference samples. A control may be any sample with a previously determined level of expression. A control may comprise material within the sample or material from sources other than the sample. Alternatively, the expression of a metabolite in a sample may be compared to a control that has a level of expression predetermined to signal or not signal a cellular or physiological characteristic. This level of expression may be derived from a single source of material including the sample itself or from a set of sources.

The sample in this method is preferably a biological sample from a subject. The term “sample” or “biological sample” is used in its broadest sense. Depending upon the embodiment of the invention, for example, a sample may comprise a bodily fluid including whole blood, serum, plasma, urine, saliva, cerebral spinal fluid, gastric fluid, semen, vaginal fluid, pulmonary fluid, tears, perspiration, mucus and the like; an extract from a cell, chromosome, organelle, or membrane isolated from a cell; a cell; a tissue; a tissue print, or any other material isolated in whole or in part from a living subject or organism. Biological samples may also include sections of tissues such as biopsy and autopsy samples, and frozen sections taken for histologic purposes such as blood, plasma, serum, sputum, stool, tears, mucus, hair, skin, and the like. Biological samples also include explants and primary and/or transformed cell cultures derived from patient tissues.

In some aspects, active EAC disease progression is identified when the quantity of the at least one metabolite in the sample from the subject is at least 1.5 times greater, at least 2.0 times greater, at least 2.5 times greater, at least 3.0 times greater, at least 3.5 times greater, at least 4.0 times greater, at least 4.5 times greater, at least 5.0 times greater, at least 5.5 times greater, at least 6.0 times greater, at least 6.5 times greater, at least 7.0 times greater, at least 7.5 times greater, or at least 8.0 times greater than that found in the at least one reference sample. In one embodiment, the quantity of the at least one metabolite in the sample from the subject is at least 2.0 times greater than that found in the at least one reference sample. In another embodiment, the quantity of the at least one metabolite in the sample from the subject is at least 4.0 times greater than that found in the at least one reference sample.

In other aspects, the quantity of the at least one metabolite in the sample from the subject is at between about 2.0 times greater and 20.0 times greater, between about 2.0 times greater and 10.0 times greater, or between about 2.0 times greater and 6.0 times greater than that found in the at least one reference sample.

In some aspects, active EAC disease progression is identified when the quantity of the at least one metabolite in the sample from the subject is 1.5 times less than, 2.0 times less than, 2.5 times less than, 3.0 times less than, 3.5 times less than, 4.0 times less than, 4.5 times less than, 5.0 times less than, 5.5 times less than, 6.0 times less than, 6.5 times less than, 7.0 times less than, 7.5 times less than, or 8.0 times less than that found in the at least one reference sample. In one embodiment, the quantity of the at least one metabolite in the sample from the subject is 1.5 times less than that found in the at least one reference sample. In another embodiment, the quantity of the at least one metabolite in the sample from the subject is 2.0 times less than that found in the at least one reference sample.

In other aspects, the quantity of the at least one metabolite in the sample from the subject is at between about 1.5 times less than and 10.0 times less than, between about 1.5 times less than and 8.0 times less than, or between about 1.5 times less than and 6.0 times less than that found in the at least one reference sample.

In certain aspects, the present invention provides a method for increasing the efficacy of a therapeutic treatment for a subject with EAC, the method comprising: determining EAC disease progression in the subject after the therapeutic treatment with a method disclosed herein; and altering the therapeutic treatment if the EAC disease progression remains active after the therapeutic treatment.

In some embodiments, the therapeutic treatment comprises surgery, endoscopic therapy, radiation therapy, chemotherapy, targeted therapy, and/or immunotherapy.

In other embodiments, altering the therapeutic treatment comprises increasing the frequency of treatment, changing from one type of therapy to another type, and/or changing the therapeutic agents and/or their dosages.

In other aspects, the present invention relates to a method for predicting EAC prognosis in a subject, the method comprising: determining EAC disease progression in the subject with a method disclosed herein; and predicting a poor EAC prognosis when active EAC disease progression is identified in the subject.

In some embodiments, active EAC disease progression is identified when the quantity of the at least one metabolite in the sample from the subject is at least 2.0 times greater than that found in the at least one reference sample.

In certain embodiments, the subject is treated with surgery. Surgery is the removal of the tumor and some surrounding healthy tissue during an operation. Surgery has traditionally been the most common treatment for esophageal cancer. However, currently, surgery is used as the main treatment only for patients with early-stage esophageal cancer. For patients with locally advanced esophageal cancer, a combination of chemotherapy and radiation therapy may be used before surgery to shrink the tumor. For people who cannot have surgery, the best treatment option is often a combination of chemotherapy and radiation therapy.

The most common surgery to treat esophageal cancer is called an esophagectomy, where the doctor removes the affected part of the esophagus and then connects the remaining healthy part of the esophagus to the stomach so that the patient can swallow normally. The stomach or part of the intestine may sometimes be used to make the connection. The surgeon also removes lymph nodes around the esophagus.

In addition to surgery to treat the disease, surgery may be used to help patients eat and relieve symptoms caused by the cancer. This is called palliative surgery. To do this, surgeons and doctors can put in a percutaneous gastrostomy or jejunostomy, also called a feeding tube, so that a person can receive nutrition directly into the stomach or intestine. This may be done before chemotherapy and radiation therapy is given to make sure that the person can eat enough food to maintain his or her weight and strength during treatment. The doctor may also create a bypass, or new pathway, to the stomach if a tumor blocks the esophagus but cannot be removed with surgery; this procedure is rarely used.

In certain embodiments, the subject is treated with endoscopic therapy. The following treatments use an endoscope to treat esophageal cancer and to manage side effects caused by the tumor. Endoscopy and dilation: This procedure expands the esophagus. It may have to be repeated if the tumor grows. Endoscopy with stent placement: This procedure uses an endoscopy to insert a stent in the esophagus. An esophageal stent is a metal, mesh device that is expanded to keep the esophagus open. Electrocoagulation: This type of palliative treatment helps kill cancer cells by heating them with an electric current. This is sometimes used to help relieve symptoms by removing a blockage caused by the tumor. Cryotherapy: This is a type of palliative treatment that uses an endoscope with a probe attached that can freeze and remove tumor tissue. It can be used to reduce the size of a tumor to help a patient swallow better.

In some embodiments, the subject is treated with radiation therapy. Radiation therapy is the use of high-energy x-rays or other particles to destroy cancer cells. A radiation therapy regimen, or schedule, usually consists of a specific number of treatments given over a set period of time. The most common type of radiation treatment is called external-beam radiation therapy, which is radiation therapy given from a machine outside the body.

When radiation treatment is given directly inside the body, it is called internal radiation therapy or brachytherapy. For esophageal cancer, this involves temporarily inserting a radioactive wire into the esophagus using an endoscope.

Proton beam therapy is being studied in clinical trials for esophageal cancer. Proton beam therapy is a type of external-beam radiation therapy that uses protons rather than x-rays. At high energy, protons can destroy cancer cells.

In one embodiment, the subject is treated with systemic therapy. Systemic therapy is the use of medication to destroy cancer cells. This type of medication is given through the bloodstream to reach cancer cells throughout the body. Common ways to give systemic therapies include an intravenous (IV) tube placed into a vein using a needle or in a pill or capsule that is swallowed (orally). The types of systemic therapies used for esophageal cancer include chemotherapy, targeted therapy, and immunotherapy. Systemic therapies may be administered individually or in combination. They can also be given as part of a treatment plan that includes surgery and/or radiation therapy.

In one aspect, the subject is treated with chemotherapy. A chemotherapy regimen, or schedule, usually consists of a specific number of cycles given over a set period of time. A patient may receive one drug at a time or a combination of different drugs given at the same time. Chemotherapy and radiation therapy are often given at the same time to treat esophageal cancer, called chemoradiotherapy.

In certain aspects, the subject is treated with a targeted therapy. Targeted therapy is a treatment that targets the cancer's specific genes, proteins, or the tissue environment that contributes to cancer growth and survival. This type of treatment blocks the growth and spread of cancer cells while limiting damage to healthy cells.

Not all tumors have the same targets. To find the most effective treatment, it may be necessary to run tests to identify the genes, proteins, and other factors in the tumor. This helps identify the most effective treatment whenever possible.

In other embodiments, the subject is treated with a targeted therapy for esophageal cancer. Such therapy includes HER2-targeted therapy. For esophageal cancer, the targeted therapy trastuzumab (Herceptin, Ogivri) may be used along with chemotherapy as a first treatment for patients with metastatic esophageal adenocarcinoma. For patients with metastatic or recurrent gastroesophageal cancer that is HER2 positive, ASCO, ASCP, and CAP recommend a combination of chemotherapy and HER2-targeted therapy.

Another targeted therapy is anti-angiogenesis therapy. The targeted therapy ramucirumab (CYRAMZA®) is also an option if first-line therapy, or the first treatments given, has not worked. Ramucirumab is a type of targeted therapy called an anti-angiogenic. It is focused on stopping angiogenesis, which is the process of making new blood vessels. Because a tumor needs the nutrients delivered by blood vessels to grow and spread, the goal of anti-angiogenesis therapies is to “starve” the tumor. Ramucirumab may be given by itself or with paclitaxel (ABRAXANE®), a type of chemotherapy.

In some embodiments, the subject is treated with immunotherapy. Immunotherapy, also called biologic therapy, is designed to boost the body's natural defenses to fight the cancer. It uses materials made either by the body or in a laboratory to improve, target, or restore immune system function. Pembrolizumab (KEYTRUDA®) is a type of immunotherapy that targets the PD-1/PD-L1 pathway. It is approved for patients when chemotherapy no longer works and if the cancer tests positive for PD-L1.

In certain aspects, the disclosed methods further comprise performing a metabolic enrichment analysis to identify enrichment of pathways and enzymes associated with metabolic reactions based on the metabolomic biosignature of EAC disease progression. In one embodiment, enrichment of a metabolic enzyme selected from formimidoyltransferase cyclodeamidase, glutamate formimidoyltransferase, urocanase, citrate synthase, aldehyde dehydrogenase, histidase, and/or L-Phenylalanine carboxy-lyase, and/or an enzyme responsible for histamine exchange, L-Tyrosine exchange, and/or alpha-N-Phenylacetyl-L-glutamine exchange indicates active EAC disease progression.

The following examples are given for purely illustrative and non-limiting purposes of the present invention.

### EXAMPLES

### Example 1. Metabolic Deregulations in Esophageal Adenocarcinoma Progression

Metabolic signatures of reprogramming in EA may distinguish disease progression and facilitate precise molecular classification of EA progression. To test this hypothesis, the following experiment was performed to 1) query the metabolome and identify metabolic deregulations in cell models of metaplasia, dysplasia, and EA; 2) validate these alterations in patient gastric fluid and plasma; and 3) identify metabolic signatures and pathways linked to EA progression.

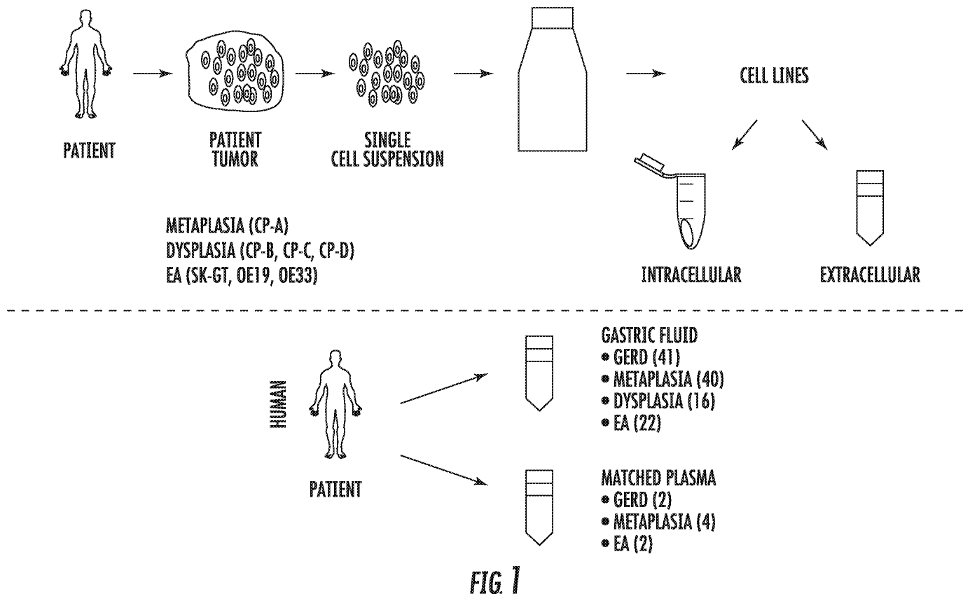

Samples were obtained from the Biobank Core Facility at St. Joseph's Hospital and Medical Center, and Barrow Neurological Institute as outlined in FIG. 1. Sample collection was conducted under an IRB-approved clinical protocol. Patients provided consent prior to collection.

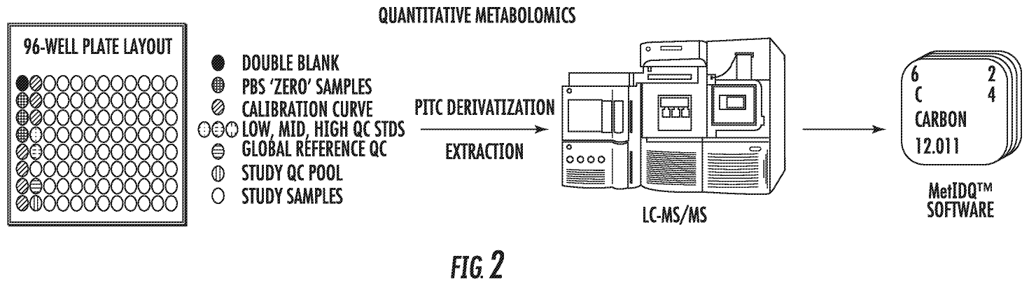

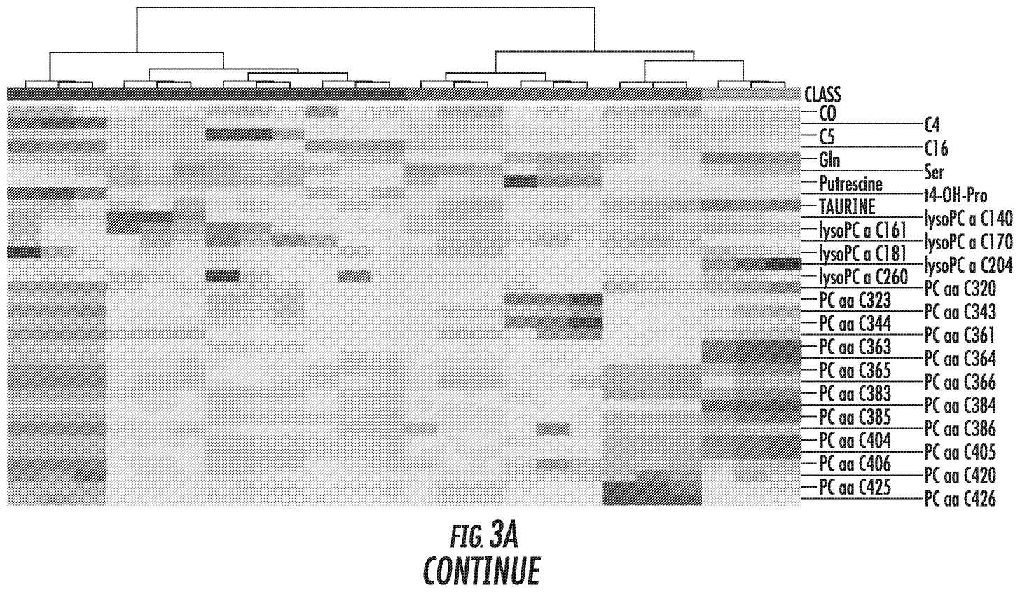

A quantitative metabolomics analysis was performed with the samples following the experimental protocol outlined in FIG. 2. For each matrix, sample pools were analyzed in triplicate to determine quality control. Only metabolites with <20% coefficient of variation were selected for further analysis.

Metabolites showing heterogeneity between the disease groups were identified by ANOVA and Tukey post hoc test (in the presence of normal distribution and homoscedasticity), or Kruskal-Wallis and Dunn post hoc test. The results of the ANOVA and Kruskal Wallis tests were adjusted with the Benjamini-Hochberg method accounting for the number of metabolites. Hierarchical clustering was conducted using the Euclidean distance and Ward clustering method. A logistic regression model was run for each metabolite in order to compute the Receive Operative Curve (ROC). Metabolites were then selected with an Area under Curve (AUC)>0.80 to estimate the AUC and ROC for a panel of metabolites able to discriminate the Dysplasia and Metaplasia groups. Analysis were conducted using the R statistical programming language.

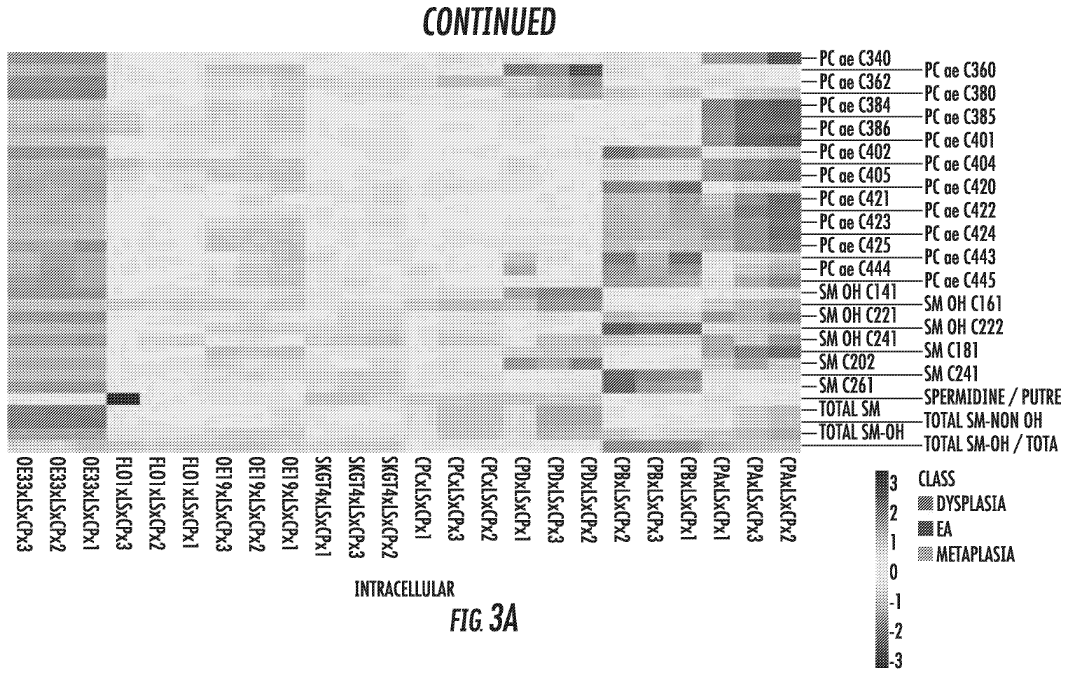

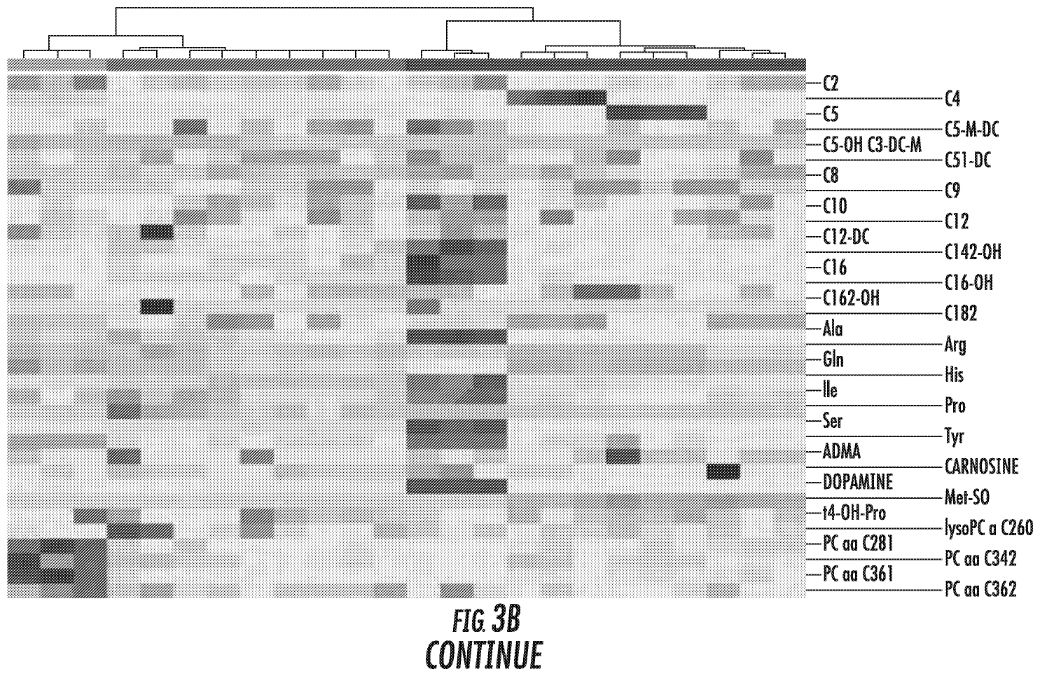

Hierarchical clustering of Metaplasia, Dysplasia and EA groups based on significant metabolite concentrations (Kruskal-Wallis test, adj. p<0.05) indicated tight clustering within each group for both intra- and extra-cellular metabolites (see FIGS. 3A and 3B). Each disease group further separated into sub-clusters suggesting metabolic heterogeneity within each disease group. Such variations contribute to metabolic phenotypes for each cell line.

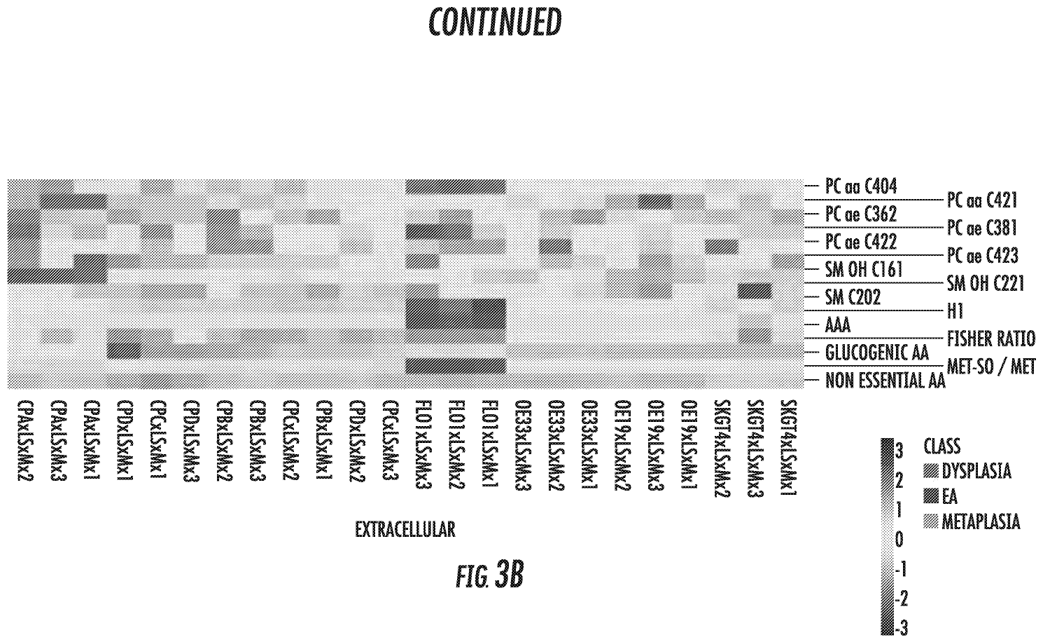

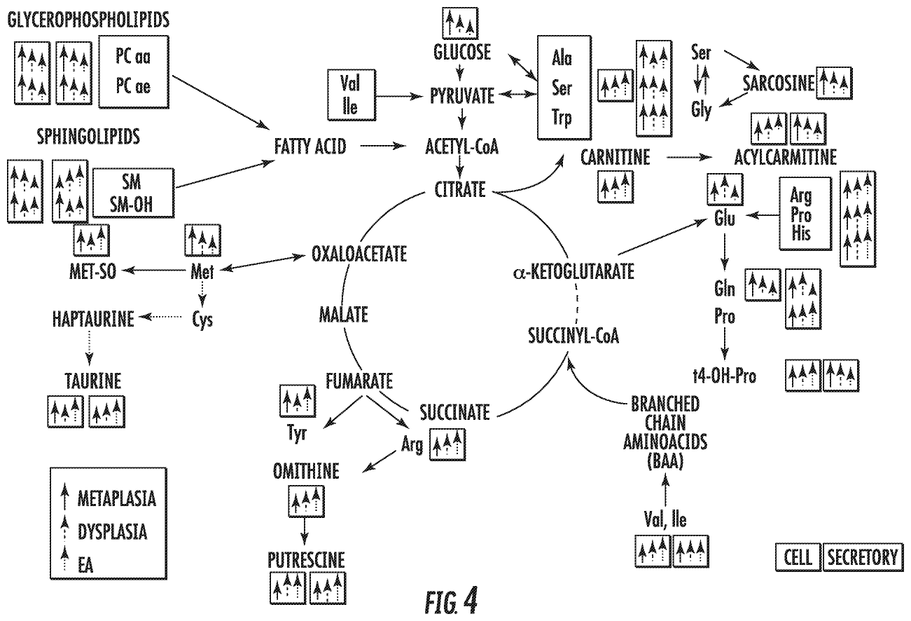

Altered metabolic pathways in the progression of esophageal adenocarcinoma are shown in FIG. 4. An intra- and extra-cellular decrease in glycerophospholipids in Dysplasia and EA suggested an increased catabolism upon EA progression. High levels of Met-SO, t4-OH-Pro taurine, ornithine, and putrescine in Dysplasia and EA highlighted increased oxidative stress upon EA progression. An increase in extracellular Arg, Pro, His, Glu, Gln, and Val was observed in Dysplasia and EA.

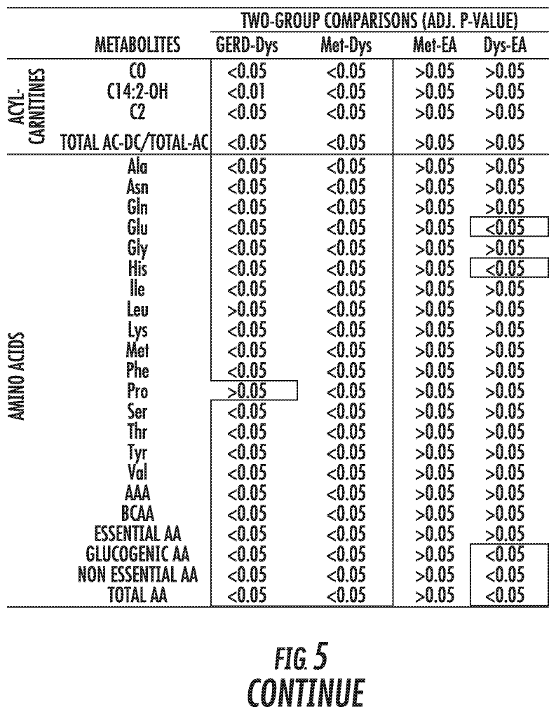

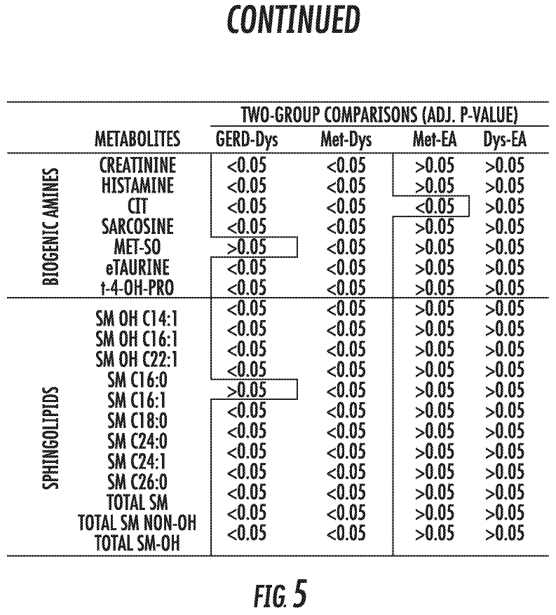

Metabolic changes occurring during the progression of esophageal adenocarcinoma are shown in FIG. 5. No significant differences were observed between GERD and Metaplasia. Acylcarnitines, amino acids, biogenic amines, and sphingolipids were significantly different (post hoc adj. p<0.05) in Dysplasia compared to GERD and Metaplasia. Hierarchical clustering (WardD2 method; sqrt transformed values) of Dysplasia and Metaplasia showed the majority of Metaplasia samples cluster together. Mixed clustering of patients may suggest variable rates of metabolic progression from Metaplasia to Dysplasia.

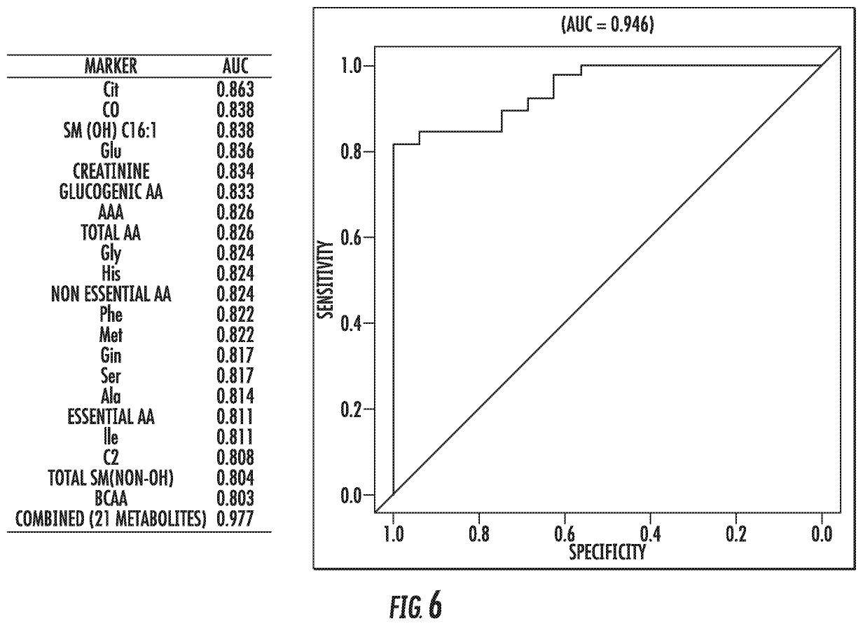

A univariate and multivariate logistic regression of the experimental data is presented in FIG. 6 along with a receiver operating curve (ROC). The logistic regression analysis revealed 21 metabolites with (AUC)>0.8. The ROC curve of a combined panel of 21 metabolites resulted in an area under the curve (AUC)=0.977.

Significant deregulations in lipid, amino acid, and biogenic amine metabolism were observed in both intracellular and extracellular models of Dysplasia and EA. Metabolic trends of progression in the extracellular metabolism of the cell models were successfully validated in gastric fluids of Dysplasia and EA patients and can serve as metabolic phenotypes describing EA progression. High levels of amino acids in gastric fluid of Dysplasia and EA corroborate with the literature. T4-OH-Pro may serve as an indicator of collagen degradation due to increased acid reflux and cellular changes in Dysplasia and EA. Met-SO, taurine, and polyamine levels in Dysplasia and EA further strengthen oxidative stress and inflammation, which play important roles in EA progression. Increased acylcarnitines and sphingolipids in gastric fluids of Dysplasia and EA patients can serve as energy sources and promote cancer cell survival and proliferation. A panel of markers consisting of amino acids, acylcarnitines and sphingolipids (AUC=0.977) was identified with the potential to differentiate Metaplasia from Dysplasia patients and monitor progression to EA.

### Example 2. Additional Analysis of Metabolic Deregulations in the Progression of Metaplasia, Dysplasia, and Esophageal Adenocarcinoma

### Materials and Methods

### Chemicals

Chemicals and solvents were purchased from Sigma-Aldrich (St. Lois, Mo.) or Fisher Scientific (San Jose, Calif.) unless otherwise stated. The chemicals used in this study were AR or MB grade, while formic acid (FA) and solvents were LC-MS grade.

### Cell Culture

Cancer cell lines used in this study included non-dysplastic metaplasia (CP-A), high-grade dysplasia (CP-B, CP-C and CP-D) and esophageal adenocarcinoma (FLO-1, SK-GT-4, OE19, OE33) cell lines. The cultures were grown in Dulbecco's modified Eagle's minimal medium with low serum (0.1% Serum) condition in a humidified atmosphere with 5% CO₂ at 37° C. until 90% cell confluence. Culture medium was removed from cells and cells were washed with cold 0.5 mM EDTA in 1× phosphate buffer saline (PBS) three time. Cell count was performed before obtaining a cell pellet. Both cell pellet and medium were immediately stored at −80° C. for further processing. All the experiments were performed in triplicates. The frozen cell pellets (3×10⁶ cells) were thawed on ice and resuspended into 75 μL of ice-cold ethanol/100 mM phosphate buffer, pH 7.5 (85:15, V/V). The cells were lysed by two cycles of sonication-freezing-sonication for 3 min-30 sec-3 min durations. The lysate was centrifuged at 18,000 rpm for 5 min at 4° C. to obtain clear supernatant for further use. The cell lysate was used for determining intracellular metabolism and culture medium was employed for extracellular metabolism.

### Human Gastric Fluid Collections

The gastric fluid samples were obtained from a total of 119 patients with GERD (41), metaplasia (40), dysplasia (16) and EAC (21) (Table 1) enrolled at St. Joseph's hospital, after approval by Institutional Review Board (IRB details). Signed informed consent was obtained from each participant. All the participants were on 12 hours fast and refrained from alcohol and any medication before sample collection. Samples were immediately stored at −80° C. after collection. This study was performed in accordance with principles set out in the Declaration of Helsinki and the Department of Health and Human Services Belmont Report.

TABLE 1
Composition of the study cohort.

| | GERD | Metaplasia | Dysplasia | EAC |
|---|---|---|---|---|
| | (41) | (40) | (16) | (21) |
| Parameters | | | | |
| Age (years) | 63.0 ± 11.1 | 63.0 ± 13.6 | 69.0 ± 7.1 | 68.0 ± 12.1 |
| BMI (kg/m² ) | 29.6 ± 5.7 | 28.9 ± 4.3 | 28.7 ± 4.1 | 26.4 ± 5.2 |
| Obese (%) | 43.9 | 37.5 | 43.8 | 25.0 |
| Overweight (%) | 24.0 | 40.0 | 50.0 | 20.0 |
| Gender* | | | | |
| Males (%) | 36.5 | 60.0 | 87.5 | 85.7 |
| Females (%) | 63.4 | 40.0 | 12.5 | 14.3 |

*Significant (p = 1.61 × 10⁻⁴), no significant difference for Age (p = 0.121) and BMI (p = 0.076) within four groups (Kruskal-Wallis test)

### Targeted Metabolomics Assay

Quantitative metabolomics was performed by measuring 185 metabolite levels in cell lines, cell culture media and gastric fluid using AbsoluteIDQ® p180 kit (Biocrates Life Sciences AG, Innsbruck, Austria). According to the manufacturer's protocol, 10 μL of gastric fluid and culture media were used, whereas for cell pellets 25 μL (1×10⁶ cells), was consumed for this assay. A portion of each sample was pooled for all 3 sample types and assayed in triplicate for sample quality control (QC). This QC pool was placed at equal distance between samples to determine the assay performance. The kit provided plasma standards with known low, medium and high concentrations of metabolites and was used for assay validation. All samples were randomized during sample preparation and data acquisition.

The data acquisition was performed on the Acquity HPLC and Xevo TQ-S mass spectrometer (Waters, Milford, Mass.) as per vendor's protocol. The targeted panel of 185 metabolites consisted of amino acids (21), biogenic amines (21), acylcarnitines (40), glycerophospholipids (89), sphingolipids (14) and hexose. Standard nomenclature for metabolites are as follows, lysophosphatidyl glycerophospholipids (Lyso PC x:y), glycerophospholipids (PC aa x:y and PC ae x:y) whereas, SM x:y and SM[OH] x:y for sphingolipids. In x:y, x is number of carbons in the side chain and y is number of unsaturated chains. PC aa consists of two fatty acids linked with a glycerol moiety, whereas PC ae contains a fatty acid and fatty alcohol bound to glycerol. Metabolite concentrations were measured in units of μM.

### Data Analysis

All the metabolites were validated using MetIDQ™ before data processing. Metabolite concentrations below 20% coefficient variation (CV) in QC pool were selected for further analysis. Only metabolites present in more than 50% population within each group were selected for data analysis and the remaining missing values were replaced by half of the minimum positive value in the original data. Principal Components Analysis (PCA) was separately conducted in each group (GERD, metaplasia, dysplasia, and EAC) aiming to detect outliers. Then, the normal distribution was assessed in each group using the Shapiro-Wilk test (30). The variance homoscedasticy was assessed between groups (pairwise comparisons) using the Levene test (31). Metabolites showing heterogeneity between the disease groups were identified by unpaired t-test (for homogeneous or heterogeneous variance according the results of the Levene test) for normally distributed variables, or with the unpaired Wilcoxon test (30, 32). Results were adjusted with the Benjamin-Hochberg method accounting for the number of metabolites (33). Hierarchical clustering was conducted using the Euclidean distance and Ward clustering method (34). A logistic regression model was run for each metabolite in order to compute the Receive Operative Curve (ROC), then selecting the metabolites with area under curve (AUC)>0.80, to estimate the AUC and ROC for the panels of metabolites able to discriminate dysplasia and metaplasia groups. All the analyses were conducted using the R statistical programming language. The metabolite set enrichment analysis was performed using OmicsNet (35).

### Results

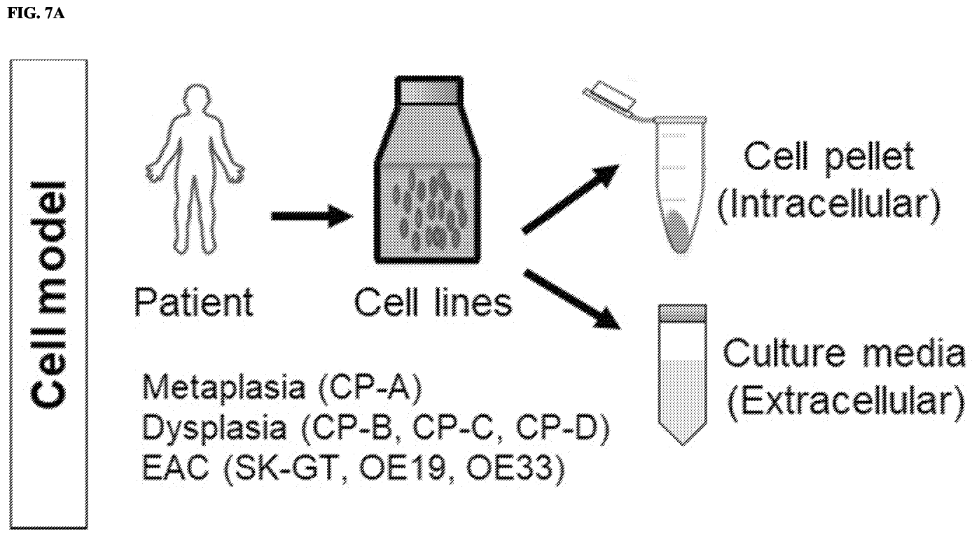

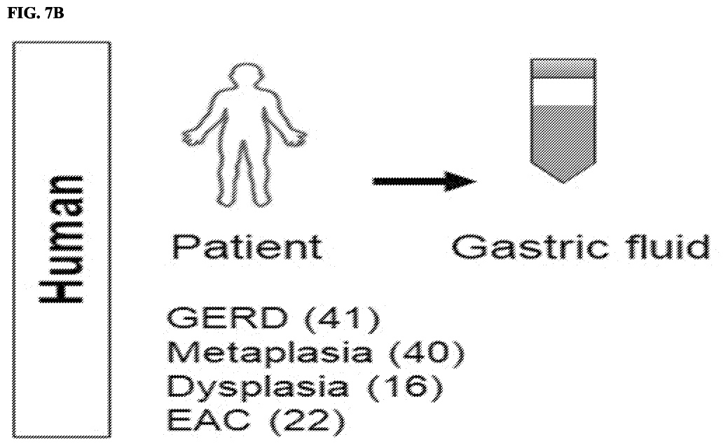

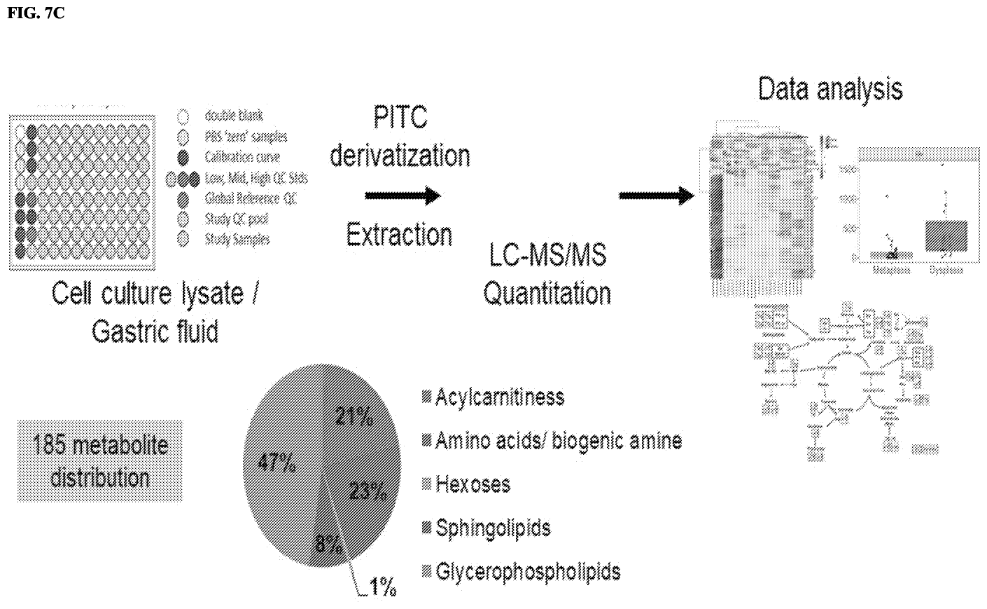

A targeted metabolomics approach was employed on cell models to understand a link between metabolic changes and esophageal disease progression which were verified in gastric fluid of patients. The experimental design and study workflow are outlined in FIGS. 7A, 7B, and 7C. The results of this study are described as follows.

### Dynamics of Intracellular and Extracellular Metabolites in Metaplasia, Dysplasia and EAC Cell Models

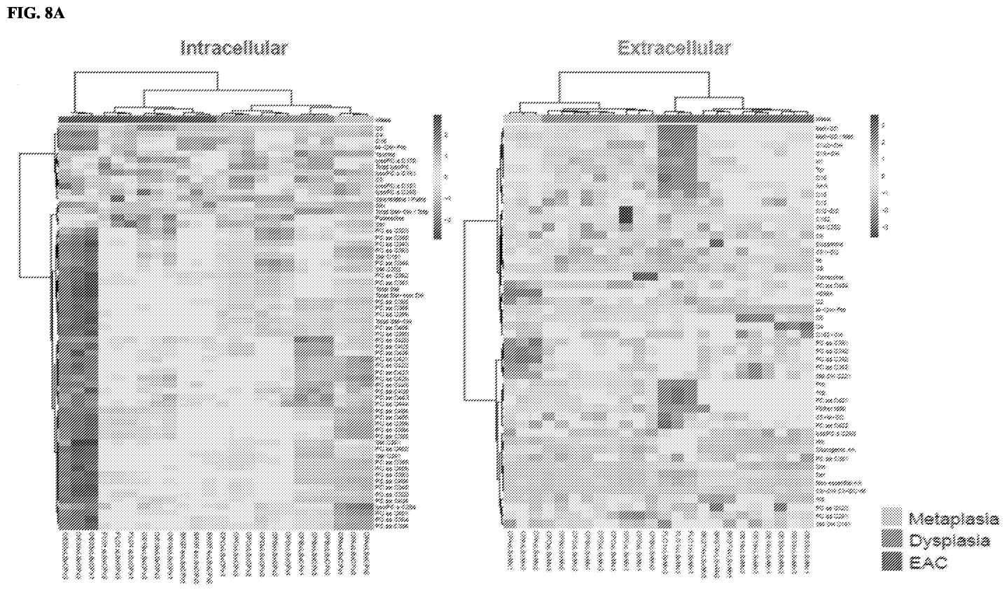

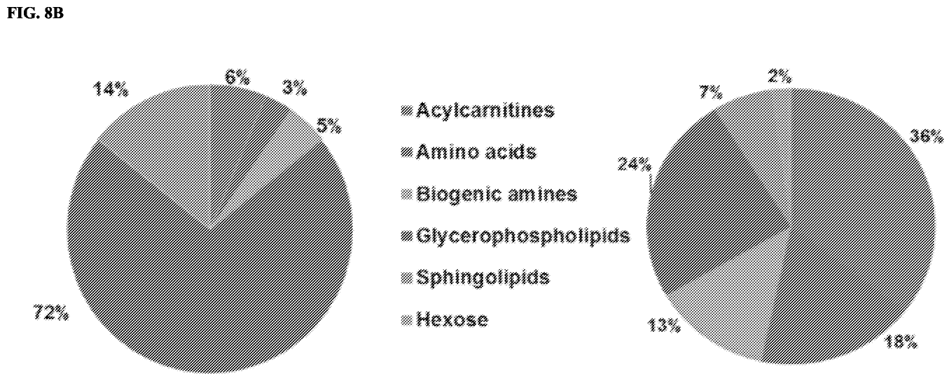

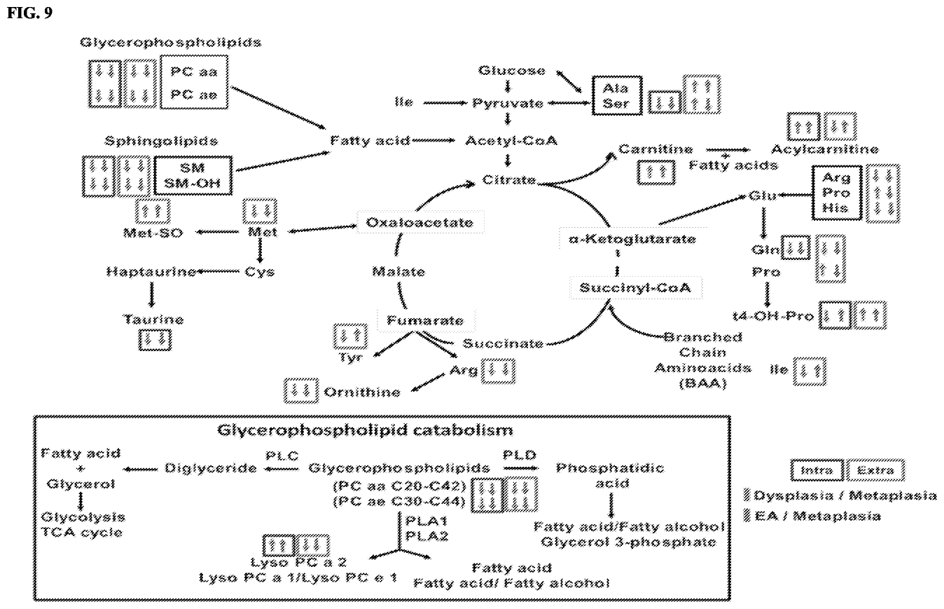

The metabolome of non-dysplastic metaplasia (herein referred as metaplasia), high grade dysplasia (dysplasia) and EAC cell lines was queried to determine how the dynamics of metabolite exchange and regulation in tumor microenvironment was affected during disease progression. Use of supervised hierarchical clustering, each cell line was grouped into its own disease stage in both intra- and extracellular metabolism (FIG. 8A). The metabolic sub-clustering of each cell type within a cluster of each disease group indicated metabolic heterogeneity at both levels. These features may contribute to metabolic phenotypes of cancer progression.

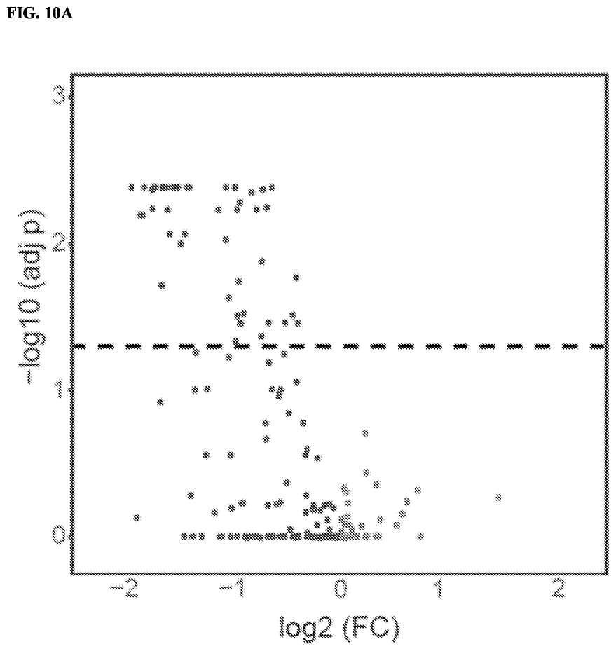

The intracellular metabolic cluster of OE33 was distinct from a large cluster that separated EAC and dysplasia-metaplasia (FIG. 8A, Left panel). Observed in the metaplasia-dysplasia cluster, CPA and CPB cell lines were closely related compared to CPD and CPC, which illustrates gradual progression from metaplasia to dysplasia. A total of 63 intracellular metabolites including 4 acylcarnitines, 2 amino acids, 3 biogenic amines, 6 lysophosphatidylcholines (lysoPC), 39 glycerophospholipids, and 9 sphingolipids were significantly different (Adj. p<0.05) between metaplasia, dysplasia and EAC (Table 2). Compared to metaplasia, the majority of the glycerophospholipids and sphingolipids were lower in dysplasia and EAC, whereas 3 lysoPCs and acylcarnitines were higher in both the groups (FIG. 8B, Left panel; Table 2). The glycerophospholipids were lower in OE33 than in any other EAC cell line. Through these models a gradual decrease in glycerophospho- and sphingolipid levels were observed from CPA (metaplasia), CPB to CPD, CPE (dysplasia) and four EAC cell lines (FIG. 8B, Left panel; FIG. 9). Along with this, amino acids Gln and Ser concentrations were progressively decreased (FIG. 9).

TABLE 2
Differentiation of intracellular metabolome in metaplasia, dysplasia and EAC.

| | Intracellular | Log2 Fold change | | | Significance | |
|---|---|---|---|---|---|---|
| Class | Metabolites | Dys/Met | EAC/DYS | EAC/Met | p value | FDR |
| Acylcarnitine | C0 | 0.01 | 0.72 | 0.73 | 0.0018 | 0.0093 |
| | C16 | −0.43 | 2.11 | 1.68 | 0.0143 | 0.0446 |
| | C4 | 0.81 | 1.43 | 2.24 | 0.0108 | 0.0372 |
| | C5 | 0.23 | 3.98 | 4.22 | 0.0123 | 0.0400 |
| Amino acid | Gln | −0.75 | −2.96 | −3.71 | 0.0001 | 0.0018 |
| | Ser | −0.14 | −1.10 | −1.24 | 0.0006 | 0.0038 |
| Biogenic | Putrescine | 0.60 | −1.50 | −0.91 | 0.0012 | 0.0068 |
| amine | t4-OH-Pro | −0.42 | 4.66 | 4.23 | 0.0158 | 0.0478 |
| | Taurine | −1.81 | 0.67 | −1.14 | 0.0061 | 0.0265 |
| | Spermidine/Putrescine | −0.78 | 1.53 | 0.75 | 0.0004 | 0.0026 |
| Glycero- | lysoPC a C16:1 | 0.54 | 1.68 | 2.23 | 0.0001 | 0.0018 |
| phospholipid | lysoPC a C17:0 | −0.23 | 0.54 | 0.31 | 0.0091 | 0.0342 |
| | lysoPC a C18:1 | 0.29 | 0.99 | 1.28 | 0.0002 | 0.0018 |
| | lysoPC a C20:4 | −2.20 | −0.48 | −2.68 | 0.0097 | 0.0351 |
| | lysoPC a C26:0 | 0.61 | 0.83 | 1.44 | 0.0108 | 0.0372 |
| | Total lysoPC | −0.47 | 0.58 | 0.11 | 0.0169 | 0.0497 |
| | PC aa C32:0 | −0.76 | −0.60 | −1.36 | 0.0102 | 0.0365 |
| | PC aa C32:3 | 0.32 | −1.47 | −1.15 | 0.0002 | 0.0018 |
| | PC aa C34:3 | −0.25 | −1.04 | −1.30 | 0.0012 | 0.0068 |
| | PC aa C34:4 | 0.20 | −1.69 | −1.48 | 0.0002 | 0.0018 |
| | PC aa C36:1 | −0.20 | −1.21 | −1.41 | 0.0012 | 0.0068 |
| | PC aa C36:3 | −1.40 | −1.19 | −2.59 | 0.0002 | 0.0021 |
| | PC aa C36:4 | −1.41 | −1.28 | −2.69 | 0.0001 | 0.0018 |
| | PC aa C36:5 | −0.42 | −1.41 | −1.83 | 0.0001 | 0.0018 |
| | PC aa C36:6 | 0.03 | −1.23 | −1.19 | 0.0002 | 0.0018 |
| | PC aa C38:3 | −0.76 | −1.06 | −1.82 | 0.0025 | 0.0114 |
| | PC aa C38:4 | −1.88 | −1.39 | −3.27 | 0.0003 | 0.0021 |
| | PC aa C38:5 | −0.78 | −1.61 | −2.40 | 0.0001 | 0.0018 |
| | PC aa C38:6 | −0.11 | −1.20 | −1.31 | 0.0003 | 0.0021 |
| | PC aa C40:4 | −1.11 | −1.14 | −2.25 | 0.0018 | 0.0093 |
| | PC aa C40:5 | −0.89 | −1.54 | −2.43 | 0.0001 | 0.0018 |
| | PC aa C40:6 | −0.02 | −1.14 | −1.17 | 0.0003 | 0.0022 |
| | PC aa C42:0 | −0.08 | −0.28 | −0.36 | 0.0160 | 0.0478 |
| | PC aa C42:5 | 0.14 | −1.33 | −1.20 | 0.0013 | 0.0071 |
| | PC aa C42:6 | 0.51 | −1.16 | −0.65 | 0.0025 | 0.0114 |
| | PC ae C34:0 | −0.92 | −0.54 | −1.46 | 0.0024 | 0.0114 |
| | PC ae C36:0 | 0.60 | −1.14 | −0.54 | 0.0002 | 0.0018 |
| | PC ae C36:2 | 0.86 | −0.83 | 0.03 | 0.0065 | 0.0271 |
| | PC ae C38:0 | 0.01 | −1.00 | −0.98 | 0.0002 | 0.0018 |
| | PC ae C38:4 | −1.88 | −0.58 | −2.45 | 0.0074 | 0.0282 |
| | PC ae C38:5 | −1.77 | −0.41 | −2.18 | 0.0121 | 0.0400 |
| | PC ae C38:6 | −1.48 | −0.26 | −1.73 | 0.0151 | 0.0466 |
| | PC ae C40:1 | −1.43 | −1.22 | −2.65 | 0.0001 | 0.0018 |
| | PC ae C40:2 | −0.02 | −1.08 | −1.11 | 0.0007 | 0.0047 |
| | PC ae C40:4 | −1.09 | −0.53 | −1.61 | 0.0097 | 0.0351 |
| | PC ae C40:5 | −1.16 | −0.55 | −1.72 | 0.0143 | 0.0446 |
| | PC ae C42:0 | 0.04 | −0.29 | −0.25 | 0.0123 | 0.0400 |
| | PC ae C42:1 | −0.73 | −0.65 | −1.38 | 0.0072 | 0.0279 |
| | PC ae C42:2 | −0.99 | −1.06 | −2.05 | 0.0006 | 0.0040 |
| | PC ae C42:3 | −0.77 | −1.25 | −2.03 | 0.0001 | 0.0018 |
| | PC ae C42:4 | −1.06 | −1.54 | −2.59 | 0.0009 | 0.0055 |
| | PC ae C42:5 | −0.36 | −0.27 | −0.63 | 0.0055 | 0.0242 |
| | PC ae C44:3 | −0.05 | −0.71 | −0.76 | 0.0124 | 0.0400 |
| | PC ae C44:4 | −0.10 | −0.62 | −0.72 | 0.0069 | 0.0279 |
| | PC ae C44:5 | −0.33 | −0.56 | −0.88 | 0.0063 | 0.0268 |
| Sphingolipid | SM C18:1 | −0.92 | −1.72 | −2.63 | 0.0001 | 0.0018 |
| | SM C20:2 | −0.24 | −1.41 | −1.65 | 0.0002 | 0.0018 |
| | SM C24:1 | 0.15 | −1.86 | −1.71 | 0.0004 | 0.0026 |
| | SM C26:1 | 0.34 | −1.11 | −0.78 | 0.0070 | 0.0279 |
| | Total SM | −0.05 | −0.92 | −0.96 | 0.0004 | 0.0026 |
| | Total SM-non OH | −0.04 | −0.85 | −0.89 | 0.0008 | 0.0051 |
| | Total SM-OH | −0.14 | −1.69 | −1.83 | 0.0001 | 0.0018 |
| | Total SM-OH/Total | −0.08 | −0.88 | −0.95 | 0.0002 | 0.0018 |
| | SM-non OH | | | | | |

p-values were determined by performing kruskal wallis test and correction (FDR) was done using Bejamini-Hochberg method, Met = Metaplasia, Dys = Dysplasia, EAC = Esophageal adenocarcinoma

To study the cellular metabolic exchange, culture media was used as a surrogate of extracellular or secretory metabolism in these cell lines. In contrast to intracellular metabolism, acylcarnitines, amino acids and biogenic amines were majorly deregulated (FIG. 8B, right panel). A total of 45 extracellular metabolites (16 acylcarnitines, 8 aminoacids, 6 biogenic amines, 11 glycerophospholipids, 3 sphingolipids and a hexose) were significantly different (Adj. p<0.05) between the three disease groups (Table 3). The extracellular glucogenic amino acids, Ser, Gln, His, Arg and Pro were down-regulated, whereas Ile, Tyr and Ala were up-regulated in EAC (FIG. 8A, Table 3). We also saw increase in Met-SO, Met-SO/Met, asymmetric dimethyl arginine (ADMA), t4-OH-Pro concentrations in both dysplasia and EAC (FIG. 9; Table 3). The Met-SO and Met-SO/Met were at highest concentration in FLO1 (EAC cell line) (FIG. 8A). Similar to intracellular metabolism, the glycerophospholipids and sphingolipids were down-regulated and acylcarnitines were up-regulated in both dysplasia and EAC (FIG. 9; Table 3).

TABLE 3
Differentiation of extracellular metabolome in metaplasia, dysplasia and EAC.

| | | Log2 Fold charge | | | Significance | |
|---|---|---|---|---|---|---|
| Class | Metabolites | Dys/Met | EAC/Dys | EAC/Met | p. value | FDR |
| Acylcarnitine | C2 | 0.24 | 0.15 | 0.39 | 0.0041 | 0.0205 |
| | C4 | 0.37 | 0.55 | 0.92 | 0.0019 | 0.0113 |
| | C5 | −0.14 | 2.11 | 1.96 | 0.0004 | 0.0034 |
| | C5:1-DC | −0.09 | 0.48 | 0.39 | 0.0005 | 0.0040 |
| | C5-M-DC | 0.08 | −0.36 | −0.28 | 0.0024 | 0.0134 |
| | C5-OH, C3-DC- | −0.11 | −4.18 | −4.30 | 0.0001 | 0.0020 |
| | M | | | | | |
| | C8 | −0.06 | 0.66 | 0.60 | 0.0001 | 0.0020 |
| | C9 | −0.05 | 0.39 | 0.34 | 0.0027 | 0.0146 |
| | C10 | −0.12 | 0.44 | 0.32 | 0.0013 | 0.0083 |
| | C12 | −0.14 | 0.24 | 0.10 | 0.0024 | 0.0134 |
| | C12-DC | 0.04 | 0.35 | 0.39 | 0.0084 | 0.0354 |
| | C14:2-OH | −0.26 | 1.05 | 0.79 | 0.0008 | 0.0057 |
| | C16 | −0.24 | 0.88 | 0.63 | 0.0003 | 0.0026 |
| | C16:2-OH | 0.04 | 0.18 | 0.23 | 0.0112 | 0.0442 |
| | C16-OH | −0.51 | 1.40 | 0.90 | 0.0004 | 0.0034 |
| | C18:2 | 0.03 | 0.18 | 0.21 | 0.0127 | 0.0490 |
| Amino Acid | Ala | 1.18 | −0.53 | 0.65 | 0.0111 | 0.0442 |
| | Arg | −0.03 | −0.48 | −0.50 | 0.0002 | 0.0020 |
| | Pro | 0.04 | −1.07 | −1.02 | 0.0002 | 0.0021 |
| | Ser | 0.17 | −3.18 | −3.01 | 0.0001 | 0.0020 |
| | Tyr | −0.63 | 2.52 | 1.89 | 0.0001 | 0.0020 |
| | Gln | −0.03 | −2.46 | −2.50 | 0.0002 | 0.0020 |
| | His | −0.22 | −1.03 | −1.25 | 0.0001 | 0.0020 |
| | Ile | −0.83 | 2.41 | 1.58 | 0.0001 | 0.0020 |
| | AAA | 2.10 | 2.90 | 4.99 | 0.0028 | 0.0148 |
| | Non essential AA | 0.07 | −1.45 | −1.38 | 0.0001 | 0.0020 |
| | Glucogenic AA | 0.53 | −1.87 | −1.34 | 0.0001 | 0.0020 |
| | Fisher ratio | 0.09 | −1.35 | −1.27 | 0.0010 | 0.0071 |
| Biogenic | Met-SO | −0.11 | 1.68 | 1.57 | 0.0002 | 0.0020 |
| amines | Met-SO/Met | 0.18 | 1.18 | 1.35 | 0.0002 | 0.0024 |
| | t4-OH-Pro | 0.94 | 6.57 | 7.52 | 0.0002 | 0.0024 |
| | ADMA | 7.17 | 1.05 | 8.23 | 0.0009 | 0.0065 |
| | Carnosine | −0.77 | 1.17 | 0.39 | 0.0054 | 0.0254 |
| | Dopamine | −0.07 | 0.45 | 0.39 | 0.0001 | 0.0020 |
| Glycero- | lysoPC a C26:0 | 0.04 | −1.11 | −1.07 | 0.0004 | 0.0035 |
| phospholipid | PC aa C28:1 | 0.17 | −0.60 | −0.42 | 0.0065 | 0.0293 |
| | PC aa C34:2 | −1.49 | −0.43 | −1.93 | 0.0073 | 0.0322 |
| | PC aa C36:1 | −0.61 | −0.18 | −0.79 | 0.0096 | 0.0394 |
| | PC aa C36:2 | −1.82 | −0.26 | −2.08 | 0.0060 | 0.0278 |
| | PC aa C40:4 | −0.82 | 0.39 | −0.43 | 0.0012 | 0.0080 |
| | PC aa C42:1 | −0.14 | −0.67 | −0.81 | 0.0001 | 0.0020 |
| | PC ae C36:2 | −0.38 | −0.20 | −0.58 | 0.0044 | 0.0212 |
| | PC ae C38:1 | −0.12 | −1.00 | −1.12 | 0.0002 | 0.0020 |
| | PC ae C42:2 | −0.27 | −0.43 | −0.71 | 0.0036 | 0.0186 |
| | PC ae C42:3 | −0.06 | −0.71 | −0.77 | 0.0080 | 0.0345 |
| | SM C20:2 | −0.65 | 1.38 | 0.73 | 0.0022 | 0.0131 |
| Sphingolipid | SM OH C16:1 | −0.22 | −0.42 | −0.64 | 0.0017 | 0.0110 |
| | SM OH C22:1 | −1.08 | −1.15 | −2.23 | 0.0001 | 0.0020 |
| Hexose | H1 | −1.05 | 1.83 | 0.78 | 0.0002 | 0.0024 |

p-values were determined by performing kruskal wallis test and correction (FDR) was done using Bejamini-Hochberg method

Association of Gastric Metabolite Deregulation with EAC Progression

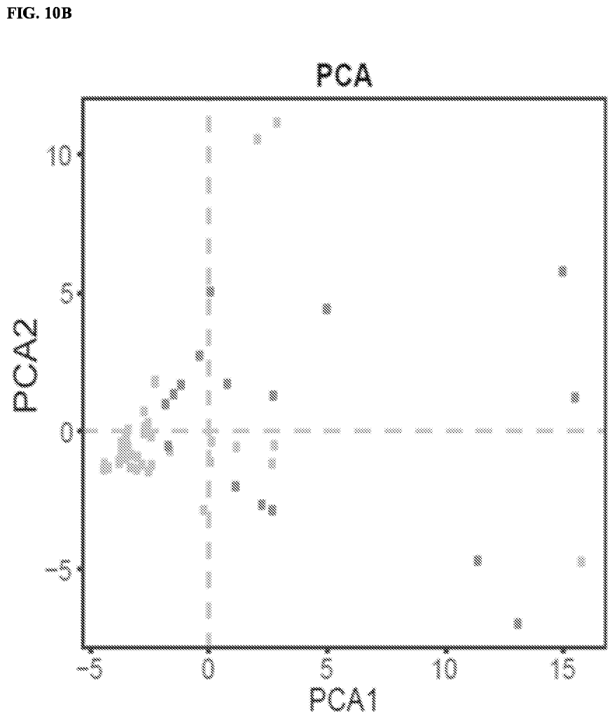

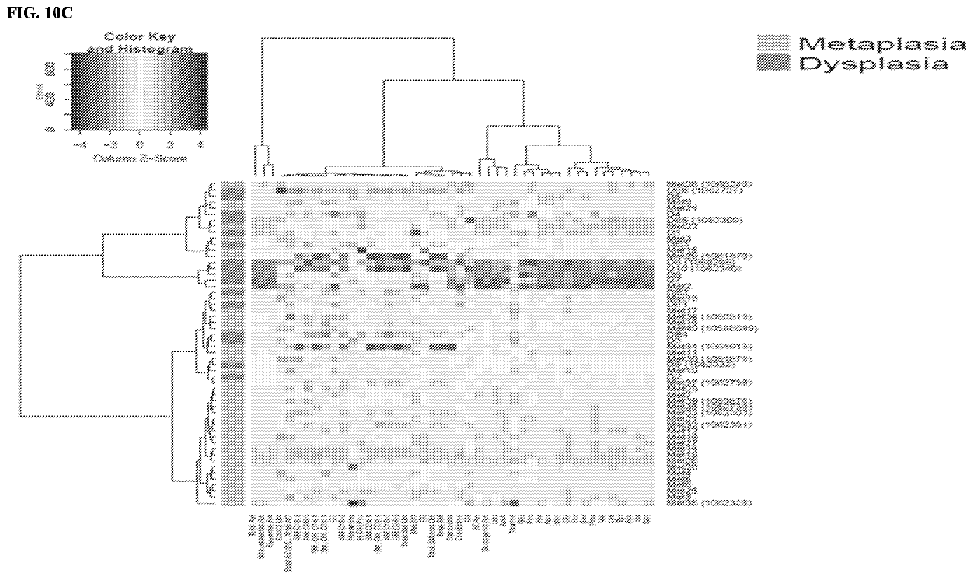

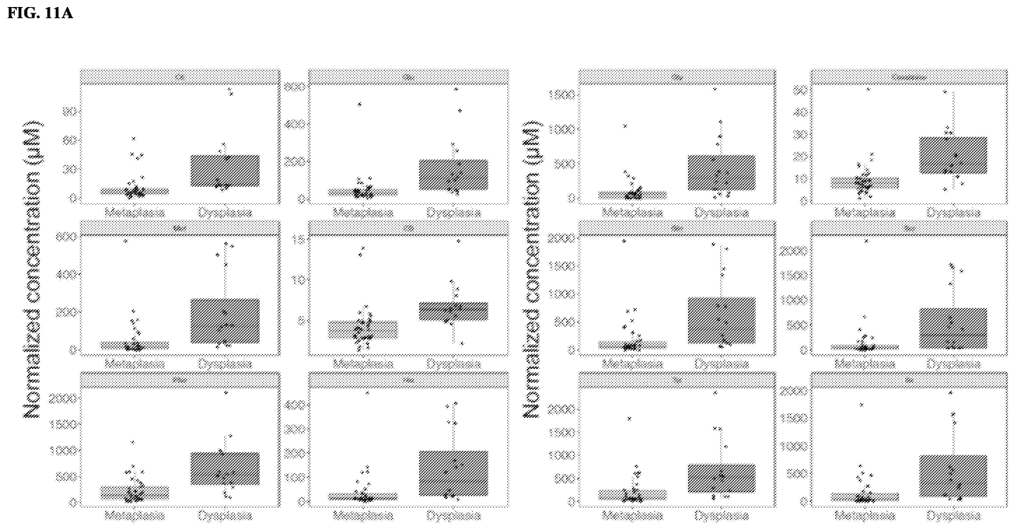

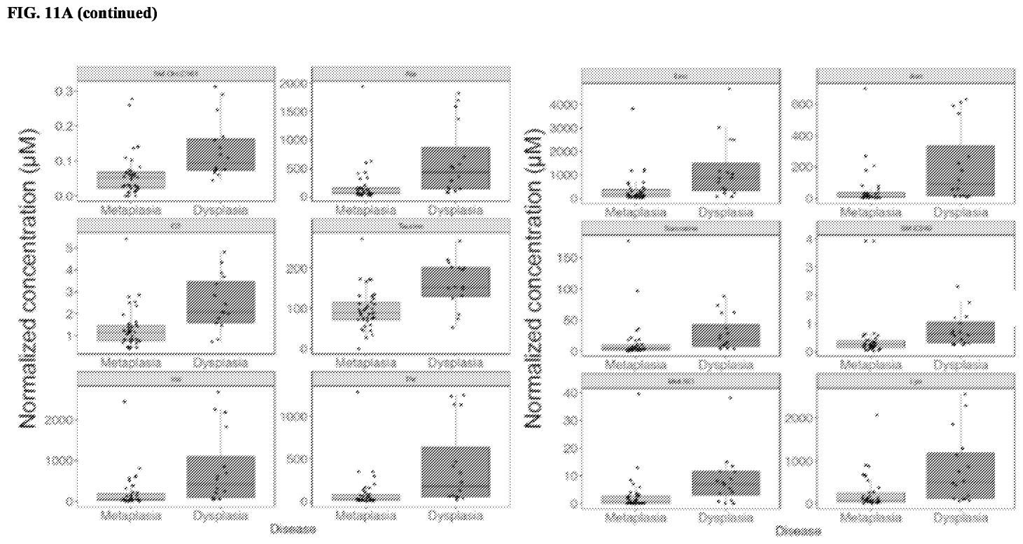

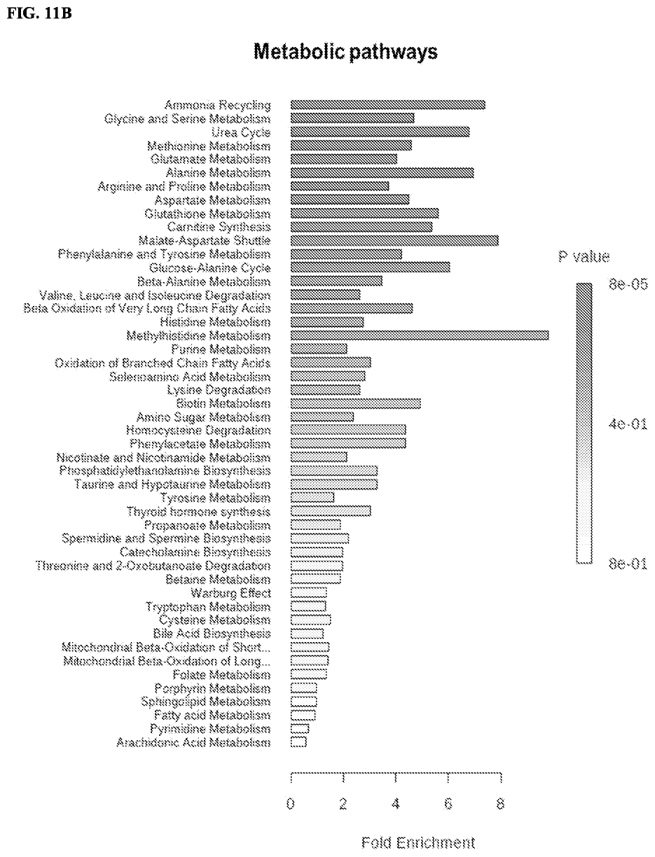

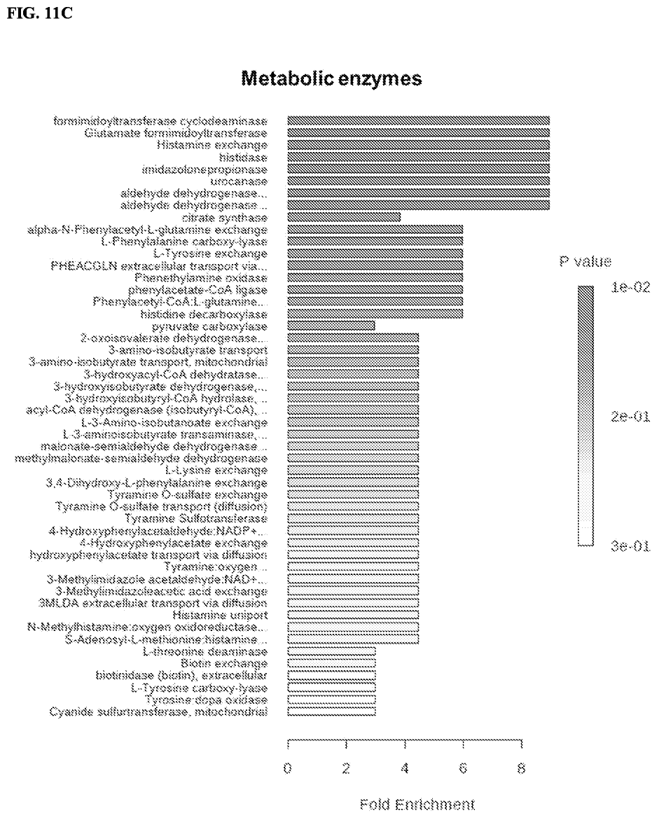

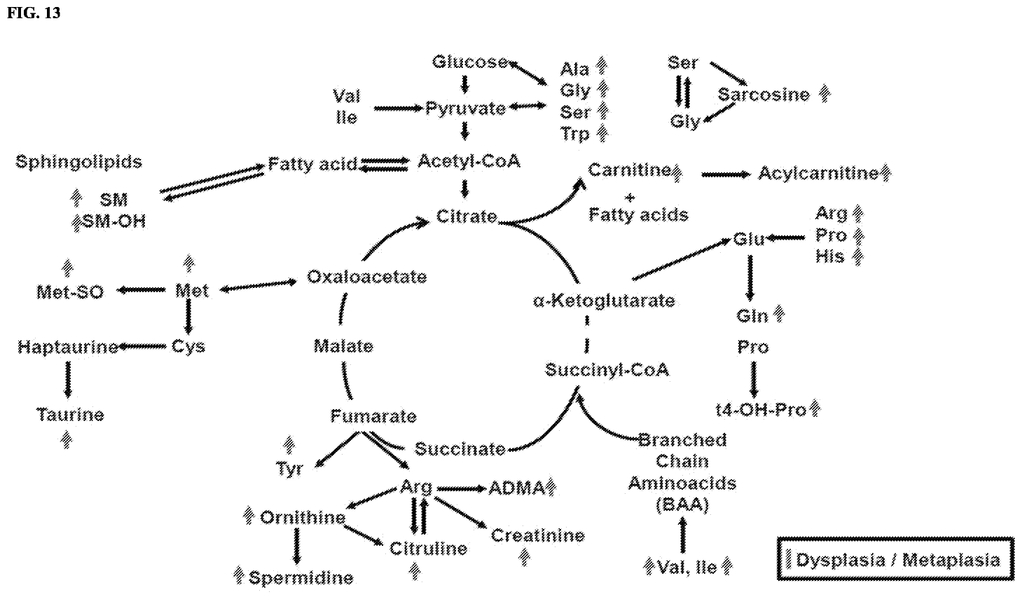

To verify the above metabolic changes, gastric fluid was employed in a cross-sectional cohort comprised of 119 patients including 41 GERD, 40 metaplasia, 16 dysplasia and 21 EAC (Table 1). We removed 5 samples from the dataset (3 GERD and 2 metaplasia), as they showed to be outliers according the PCA analysis. No significant difference was observed in the age and basal metabolic rate between these diseases groups. However, ratio of males to females was significantly higher (p<0.05) at advanced stages of cancer (dysplasia and EAC). Upon comparison, no significant difference in metabolite concentrations was observed between GERD and metaplasia. As with the cell models, we also compared dysplasia and EAC groups with metaplasia. A total of 44 metabolites including 17 amino acids, 8 biogenic amines, 9 acylcarnitines and 10 sphingolipids were found to be significantly higher in dysplasia (Adj. p<0.05) (Table 4; FIG. 10A). The PCA and hierarchical clustering using these significant metabolites showed variation within disease groups which may be regarded as metabolic subtypes of the metaplasia and dysplasia (FIGS. 10B and 10C). The concentrations of these metabolites were significantly elevated in dysplasia patients (FIG. 11A). The metabolic enrichment analysis was performed on 35 potential markers to identify enrichment of pathways and enzymes associated with metabolic reactions. The metabolism of various amino acids, urea cycle, and carnitine synthesis were among the top 10 significant metabolic pathways enriched in this dataset (FIG. 11B). The top 10 significant metabolic enzymes included formimidoyltransferase cyclodeamidase, glutamate formimidoyltransferase, histamine exchange, urocanase, citrate synthase, aldehyde dehydrogenase, alpha-N-Phenylacetyl-L-glutamine exchange, histidase, L-Phenylalanine carboxy-lyase, L-Tyrosine exchange (FIG. 11C). Overall, amino acid metabolism was found to be heavily altered in the gastric fluid metabolome of dysplasia patients (FIG. 13).

TABLE 4
The gastric fluid metabolic differences between
metaplasia and dysplasia patients.

| | Log2 Fold change | | |
|---|---|---|---|
| Metabolites | Dys/Met | p. value | FDR |
| Cit | 2.7551 | 5 7E−05 | 6 4E−03 |
| Glu | 2.9989 | 1 7E−04 | 6 4E−03 |
| Met | 3.4811 | 3 2E−04 | 6 4E−03 |
| C0 | 1.4837 | 3 3E−04 | 6 4E−03 |
| Phe | 2.7015 | 3 4E−04 | 6 4E−03 |
| His | 3.3727 | 3 6E−04 | 6 4E−03 |
| Gly | 3.6667 | 3 8E−04 | 6 4E−03 |
| Creatinine | 1.7938 | 4 2E−04 | 6 4E−03 |
| Gln | 3.3134 | 4 2E−04 | 6 4E−03 |
| Ser | 3.6021 | 4 2E−04 | 6 4E−03 |
| Tyr | 3.0386 | 4 3E−04 | 6 4E−03 |
| Ile | 3.5537 | 4 8E−04 | 6 4E−03 |
| SM (OH) C16:1 | 1.7521 | 5 0E−04 | 6 4E−03 |
| Ala | 3.154 | 5 1E−04 | 6 4E−03 |
| C2 | 1.7038 | 9 9E−04 | 1 0E−02 |
| Taurine | 1.6195 | 9 9E−04 | 1 0E−02 |
| Val | 3.5853 | 1 0E−03 | 1 0E−02 |
| Thr | 3.8803 | 1 1E−03 | 1 0E−02 |
| Leu | 3.105 | 1 1E−03 | 1 0E−02 |
| Asn | 3.9002 | 1 2E−03 | 1 1E−02 |
| Sarcosine | 1.9927 | 1 3E−03 | 1 1E−02 |
| SM C24:0 | 1.3537 | 1 6E−03 | 1 3E−02 |
| Met-SO | 2.9003 | 1 7E−03 | 1 3E−02 |
| Lys | 2.9429 | 2 1E−03 | 1 5E−02 |
| SM C16:0 | 1.5794 | 2 2E−03 | 1 6E−02 |
| SM (OH) C22:1 | 1.4513 | 2 9E−03 | 1 9E−02 |
| Pro | 2.8533 | 2 9E−03 | 1 9E−02 |
| SM C24:1 | 1.2312 | 3 2E−03 | 2 0E−02 |
| Asp | 3.5721 | 3 4E−03 | 2 1E−02 |
| SM C18:0 | 1.6254 | 4 5E−03 | 2 6E−02 |
| t4-OH-Pro | 1.6401 | 4 8E−03 | 2 7E−02 |
| Histamine | 1.342 | 7 0E−03 | 3 8E−02 |
| C3-DC, C4-OH | 1.3037 | 7 4E−03 | 3 9E−02 |
| SM C26:0 | 1.8946 | 7 7E−03 | 3 9E−02 |
| C14:2-OH | 1.6607 | 8 0E−03 | 4 0E−02 |

p-values were determined by performing Wilcoxon test and t-test, correction (FDR) was done using Bejamini-Hochberg method, Data distribution for all the significant metabolites (adj p < 0.05) was non-normal. For non-normal data Wilcoxon test was performed.

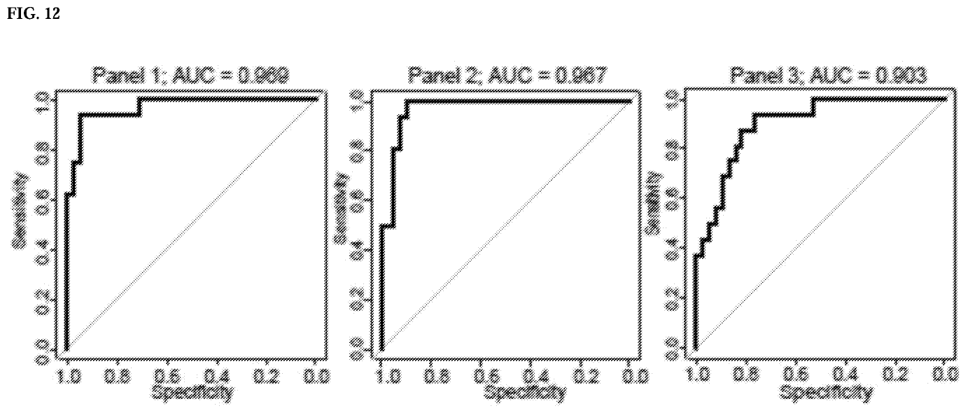

These significant metabolites were further subjected to logistic regression to determine their predictive performance in discriminating metaplasia versus dysplasia. The ROC curve analysis identified 35 of 44 metabolites with the AUC ranging from 0.72 to 0.863 (Table 5). These 35 putative markers were further segmented into three models to predict their diagnostic utility (Table 6). Compared to individual metabolites, the metabolite panels predicted better sensitivity and accuracy with AUC>0.9 (FIG. 12; Table 6). Model 1, a panel of 17 amino acids, provided highest AUC of 0.969 and thereby predicted higher diagnostic accuracy and greater power to discriminate dysplasia from metaplasia patients.

TABLE 5
Receiver operating curve analysis.

| Marker | Area under curve (AUC) |
|---|---|
| Cit | 0.863 |
| C0 | 0.838 |
| SM (OH) C16:1 | 0.838 |
| Glu | 0.836 |
| Creatinine | 0.834 |
| His | 0.824 |
| Gly | 0.824 |
| Phe | 0.822 |
| Met | 0.822 |
| Gln | 0.817 |
| Ser | 0.817 |
| Tyr | 0.815 |
| Ala | 0.814 |
| Ile | 0.811 |
| SM C24:0 | 0.810 |
| C2 | 0.808 |
| SM C16:0 | 0.803 |
| Taurine | 0.799 |
| Val | 0.798 |
| SM-OH C22:1 | 0.794 |
| Leu | 0.793 |
| Sarcosine | 0.793 |
| SM C24:1 | 0.793 |
| Thr | 0.789 |
| Asn | 0.789 |
| Lys | 0.780 |
| Met-SO | 0.777 |
| SM C18:0 | 0.776 |
| Pro | 0.774 |
| t4-OH Pro | 0.765 |
| C3-DC C4-OH | 0.758 |
| SM C26:0 | 0.756 |
| Asp | 0.753 |
| C14:2-OH | 0.747 |
| Histamine | 0.725 |

TABLE 6
Receiver operating curve analysis.

| Model | Metabolite panel | AUC |
|---|---|---|
| Model 1 | Glu, Gly, His, Phe, Met, Ser, Gln, Tyr, | 0.969 |
| | Ala, Ile, Val, Leu, Thr, Asn, | |
| | Lys, Pro, Asp | |
| Model 2 | All markers | 0.967 |
| Model 3 | Taurine, Met, Met-SO, t4-OH-Pro | 0.91 |

In exploratory analysis (un-adjusted p<0.05) the following comparisons were made. The Trp, putrescine, spermine, and spermidine were elevated along with 6 acylcarnitines and 3 sphingolipids in dysplasia in comparison to metaplasia (Table 7). The 10 metabolites including amino acids (Asn, Tyr), biogenic amines (histamine, creatinine, spermidine), acylcarnitines (C2, C5) and sphingolipids (SM C18:0, SM C26:0) were down-regulated (p<0.05), while one glycerophospholipid (PC aa C42:1) was increased (p<0.05) in dysplasia compared to EAC (Table 8). Further comparison of EAC with metaplasia also identified 7 metabolites (citrulline, ornithine, taurine, PC aa C42:1, Lyso PC a C17:0, C16-OH, SM (OH) C22:2) to be significantly up-regulated in EAC (p<0.05) (Table 9).

TABLE 7
Exploratory analysis of metabolites significantly different
between gastric fluid of dysplasia and metaplasia patients.

| Metabolites | log2(Dys/Met) | p | BH |
|---|---|---|---|
| Trp | 1.3197 | 0.0142 | 0.0550 |
| C4 | 0.4955 | 0.0149 | 0.0567 |
| C14:1-OH | 0.6947 | 0.0160 | 0.0595 |
| SM (OH) C24:1 | 0.4961 | 0.0179 | 0.0651 |
| Spermidine | 0.3864 | 0.0246 | 0.0879 |
| SM (OH) C22:2 | 0.2687 | 0.0283 | 0.0982 |
| SM C26:1 | 0.5987 | 0.0286 | 0.0982 |
| C16-OH | 1.0864 | 0.0295 | 0.0992 |
| C10:2 | 0.1530 | 0.0300 | 0.0992 |
| Putrescine | 0.5522 | 0.0323 | 0.1047 |
| Spermine | 0.5607 | 0.0345 | 0.1096 |
| C14:1 | 0.9381 | 0.0385 | 0.1204 |
| C8 | 0.3610 | 0.0466 | 0.1431 |

p-values were determined by performing Wilcoxon test and t-test, correction (FDR) was done using Bejamini-Hochberg method, Data distribution for all the significant metabolites (adj p < 0.05) was non-normal. For non-normal data Wilcoxon test was performed.

TABLE 8
Exploratory analysis of metabolites significantly different
between gastric fluid of dysplasia and EAC patients.

| Metabolites | p | BH |
|---|---|---|
| Histamine | 1.9E−02 | 6.1E−01 |
| Creatinine | 2.1E−02 | 6.1E−01 |
| SM C18:0 | 3.5E−02 | 6.1E−01 |
| Glu | 3.8E−02 | 6.1E−01 |
| His | 4.0E−02 | 6.1E−01 |
| C5 | 4.3E−02 | 6.1E−01 |
| Spermidine | 4.4E−02 | 6.1E−01 |
| SM C26:0 | 4.6E−02 | 6.1E−01 |
| C2 | 4.6E−02 | 6.1E−01 |
| PC aa C42:1 | 4.6E−02 | 6.1E−01 |

p-values were determined by performing Wilcoxon test and t-test, correction (FDR) was done using Bejamini-Hochberg method, Data distribution for all the significant metabolites (adj p < 0.05) was non-normal. For non-normal data Wilcoxon test was performed.

TABLE 9
Exploratory analysis of metabolites significantly different
between gastric fluid of dysplasia and EAC patients.

| Metabolites | Statistic | p | BH |
|---|---|---|---|
| Histamine | 254.0 | 1.9E−02 | 6.1E−01 |
| Creatinine | 254.5 | 2.1E−02 | 6.1E−01 |
| SM C18:0 | 248.0 | 3.5E−02 | 6.1E−01 |
| Glu | 246.5 | 3.8E−02 | 6.1E−01 |

### Discussion

Metabolic reprogramming is a common feature of cancer cells that acquires nutrients to support rapid proliferation, growth, survival, invasion and metastasis (13-15, 21). Deconvoluting this unique feature of metabolism has potential in early detection, monitoring and treatment of EAC. In order to understand metabolic reprogramming during EAC progression, we measured cellular uptake and release of 185 metabolites through a targeted approach in non-dysplastic metaplasia, high-grade dysplasia and EAC cell lines. In comparison to metaplasia, the intracellular level of 19 diacyl glycerophospholipids and 20 glycerophosphocholine plasmalogens were lower in dysplasia and EAC, while Lyso PCs and acylcarnitines were higher in dysplasia and EAC. A trend of increase in fatty acid content and reduction in glycerophospholipids were also observed at the extracellular level in dysplasia and EAC. All these metabolic events may point towards the catabolic fate of phospholipids through up-regulation of phospholipase A1 (PLA1), A2 (PLA2) and D (PLD) activity (FIG. 9). The PLA1 and PLA2 cleave glycerophospholipids into to Lyso PC and fatty acid or fatty alcohol, whereas the PLD forms phosphatidic acid and choline as products. The over expression of secretory PLA2 has been reported in Barrett's adenocarcinoma and several other digestive tract cancers (36-38). This suggests a possible role of altered phospholipid metabolism in cellular transformation from metaplastic cell to adenocarcinoma by increasing inflammation through elevated cyclooxygenase activity (FIG. 9).

The depletion of extracellular glucogenic amino acids such as, Ser, Gln, His, Arg and Pro in EAC may indicate their fate in glucose synthesis and energy production. The large depletion in serum amino acids were reported in esophageal cancer (39). An increase in intracellular glutaminolysis and fatty acid content was seen in both dysplasia and EA as reported by Zhu et al. in esophageal cancer tissue (39). The elevated levels of intracellular putrescine in dysplasia and spermidine/putrescine in EA indicate altered polyamine biosynthesis, as observed in many cancers (40). The increase in extracellular reactive oxygen species (ROS) indicators (Met-SO, Met-SO/Met, ADMA) (41, 42) may imply oxidative damage associated with EAC carcinogenesis as one of the hallmarks of cancer (43, 44).

This study has identified the utility of gastric fluid in distinguishing metaplasia to dysplasia. Major deregulation in amino acid metabolism was observed (FIG. 13) where 17 out 20 amino acids were significantly increased in dysplasia. These deregulated metabolites may have a fate in energy metabolism either via gluconeogenesis, glycolysis, fatty acid or sphingolipid synthesis (FIG. 13) Based on these findings is is proposed that exposure of the acidic medium and serine proteases through gastric fluid evokes inflammation and cellular stress in the lower esophagus. This may induce expression of matrix metalloproteinases (MMPs) which can alter integrity of extracellular matrix (ECM) and basement membrane, and transform metaplastic cells to dysplastic cells.

The increase in multiple amino acids in gastric fluid may have resulted from ECM degradation due to overexpressed MMP. The MMPs are zinc-dependent proteases associated with ECM and basement membrane degradation. They induce angiogenesis and are involved in growth, differentiation, diffusion and migration of tumor cells (45). Studies have shown an association between MMP expression and esophageal cancer (45-47). The link between MMPs and poor overall survival in esophagus squamous cell cancer patients has been identified previously (45-47).

The increase in gastric acylcarnitines, sphingolipids, Met-SO and t4-OH-Pro further corroborates the present finding with extracellular metabolism in cell lines. The increase in ROS scavengers such as Met-SO, taurine and elevated polyamines including putrescine, spermine, spermidine may further suggest increased oxidative stress and inflammation in gastric fluid of dysplasia patients. T4-OH-Pro, an indicator of collagen degradation, may provide association with increased acid reflux and cellular changes in dysplasia. Based on the logistic regression analysis, model 1 comprised of a combined panel of 17 amino acids predicted the highest sensitivity and accuracy with AUC 0.969. This panel allows one to stratify metaplasia and dysplasia and to identify a patient sub-population which may not have been classified correctly based on clinical pathology.

In conclusion, the study provides evidence of metabolic deregulations and their association with development of EAC. The results demonstrate the utility of gastric fluid in identifying metabolic gradation from metaplasia to dysplasia, which will have a significant impact in screening and surveillance of disease progression. The potential of these biomarkers in accurately identifying dysplasia patients will be confirmed in a larger cohort to validate the utility of gastric fluid as a minimally invasive source for improving the diagnostic evaluation and monitoring of patients at risk of EAC.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

1. (2018) Esophageal Cancer: Statistics.
2. The American Cancer Society, A., Ga (2019) American Cancer Society. Cancer Facts & Figures 2019. 76
3. Zhang, Y. (2013) Epidemiology of esophageal cancer. World journal of gastroenterology 19, 5598-5606
4. Lepage, C., Drouillard, A., Jouve, J. L., and Faivre, J. (2013) Epidemiology and risk factors for oesophageal adenocarcinoma. Dig Liver Dis 45, 625-629
5. Verbeek, R. E., Leenders, M., Ten Kate, F. J., van Hillegersberg, R., Vleggaar, F. P., van Baal, J. W., van Oijen, M. G., and Siersema, P. D. (2014) Surveillance of Barrett's esophagus and mortality from esophageal adenocarcinoma: a population-based cohort study. The American journal of gastroenterology 109, 1215-1222
6. Rastogi, A., Puli, S., El-Serag, H. B., Bansal, A., Wani, S., and Sharma, P. (2008) Incidence of esophageal adenocarcinoma in patients with Barrett's esophagus and high-grade dysplasia: a meta-analysis. Gastrointest Endosc 67, 394-398
7. Konda, V. J., Ross, A. S., Ferguson, M. K., Hart, J. A., Lin, S., Naylor, K., Noffsinger, A., Posner, M. C., Dye, C., Cislo, B., Stearns, L., and Waxman, I. (2008) Is the risk of concomitant invasive esophageal cancer in high-grade dysplasia in Barrett's esophagus overestimated? Clin Gastroenterol Hepatol 6, 159-164
8. Vaughan, T. L., and Fitzgerald, R. C. (2015) Precision prevention of oesophageal adenocarcinoma. Nat Rev Gastroenterol Hepatol 12, 243-248
9. Fitzgerald, R. C., di Pietro, M., Ragunath, K., Mg, Y., Kang, J. Y., Watson, P., Trudgill, N., Patel, P., Kaye, P. V., Sanders, S., O'Donovan, M., Bird-Lieberman, E., Bhandari, P., Jankowski, J. A., Attwood, S., Parsons, S. L., Loft, D., Lagergren, J., Moayyedi, P., Lyratzopoulos, G., de Caestecker, J., and British Society of, G. (2014) British Society of Gastroenterology guidelines on the diagnosis and management of Barrett's esophagus. Gut 63, 7-42
10. Corley, D. A., Mehtani, K., Quesenberry, C., Zhao, W., de Boer, J., and Weiss, N. S. (2013) Impact of endoscopic surveillance on mortality from Barrett's esophagus-associated esophageal adenocarcinomas. Gastroenterology 145, 312-319 e311
11. di Pietro, M., Canto, M. I., and Fitzgerald, R. C. (2018) Endoscopic Management of Early Adenocarcinoma and Squamous Cell Carcinoma of the Esophagus: Screening, Diagnosis, and Therapy. Gastroenterology 154, 421-436
12. Phan, L. M., Yeung, S. C., and Lee, M. H. (2014) Cancer metabolic reprogramming: importance, main features, and potentials for precise targeted anti-cancer therapies. Cancer Biol Med 11, 1-19
13. Ward, P. S., and Thompson, C. B. (2012) Metabolic reprogramming: a cancer hallmark even warburg did not anticipate. Cancer Cell 21, 297-308
14. Tarrado-Castellarnau, M., de Atauri, P., and Cascante, M. (2016) Oncogenic regulation of tumor metabolic reprogramming. Oncotarget 7, 62726-62753
15. El-Bacha, T., de Freitas, M. S., and Sola-Penna, M. (2003) Cellular distribution of phosphofructokinase activity and implications to metabolic regulation in human breast cancer. Mol Genet Metab 79, 294-299
16. Menendez, J. A., and Lupu, R. (2007) Fatty acid synthase and the lipogenic phenotype in cancer pathogenesis. Nat Rev Cancer 7, 763-777
17. Fujigaki, S., Nishiumi, S., Kobayashi, T., Suzuki, M., Iemoto, T., Kojima, T., Ito, Y., Daiko, H., Kato, K., Shouji, H., Honda, K., Azuma, T., and Yoshida, M. (2018) Identification of serum biomarkers of chemoradiosensitivity in esophageal cancer via the targeted metabolomics approach. Biomark Med 12, 827-840
18. Rosario, S. R., Long, M. D., Affronti, H. C., Rowsam, A. M., Eng, K. H., and Smiraglia, D. J. (2018) Pan-cancer analysis of transcriptional metabolic dysregulation using The Cancer Genome Atlas. Nat Commun 9, 5330
19. Basetti, M. (2017) Special Issue: Cancer Metabolism. Metabolites 7
20. Pavlova, N. N., and Thompson, C. B. (2016) The Emerging Hallmarks of Cancer Metabolism. Cell Metab 23, 27-47
21. Gupte, A., and Mumper, R. J. (2009) Elevated copper and oxidative stress in cancer cells as a target for cancer treatment. Cancer Treat Rev 35, 32-46
22. Park, J. K., Coffey, N. J., Limoges, A., and Le, A. (2018) The Heterogeneity of Lipid Metabolism in Cancer. Advances in experimental medicine and biology 1063, 33-55
23. Buas, M. F., Gu, H., Djukovic, D., Zhu, J., Onstad, L., Reid, B. J., Raftery, D., and Vaughan, T. L. (2017) Candidate serum metabolite biomarkers for differentiating gastroesophageal reflux disease, Barrett's esophagus, and high-grade dysplasia/esophageal adenocarcinoma. Metabolomics 13
24. Sanchez-Espiridion, B., Liang, D., Ajani, J. A., Liang, S., Ye, Y., Hildebrandt, M. A., Gu, J., and Wu, X. (2015) Identification of Serum Markers of Esophageal Adenocarcinoma by Global and Targeted Metabolic Profiling. Clin Gastroenterol Hepatol 13, 1730-1737 e1739
25. Reed, M. A., Singhal, R., Ludwig, C., Carrigan, J. B., Ward, D. G., Taniere, P., Alderson, D., and Gunther, U. L. (2017) Metabolomic Evidence for a Field Effect in Histologically Normal and Metaplastic Tissues in Patients with Esophageal Adenocarcinoma. Neoplasia 19, 165-174
26. Yang, Y., Wang, L., Wang, S., Liang, S., Chen, A., Tang, H., Chen, L., and Deng, F. (2013) Study of metabonomic profiles of human esophageal carcinoma by use of high-resolution magic-angle spinning 1H NMR spectroscopy and multivariate data analysis. Analytical and bioanalytical chemistry 405, 3381-3389
27. Wu, H., Xue, R., Lu, C., Deng, C., Liu, T., Zeng, H., Wang, Q., and Shen, X. (2009) Metabolomic study for diagnostic model of oesophageal cancer using gas chromatography/mass spectrometry. Journal of chromatography. B, Analytical technologies in the biomedical and life sciences 877, 3111-3117
28. Miyashita, T., Miwa, K., Fujimura, T., Ninomiya, I., Fushida, S., Shah, F. A., Harmon, J. W., Hattori, T., and Ohta, T. (2013) The severity of duodeno-esophageal reflux influences the development of different histological types of esophageal cancer in a rat model. Int J Cancer 132, 1496-1504
29. Miyashita, T., Ohta, T., Fujimura, T., Ninomiya, I., Fushida, S., Hattori, T., and Miwa, K. (2006) Duodenal juice stimulates oesophageal stem cells to induce Barrett's esophagus and oesophageal adenocarcinoma in rats. Oncology reports 15, 1469-1475
30. David, F. N., and Johnson, N. L. (1951) The effect of non-normality on the power function of the F-test in the analysis of variance. Biometrika 38, 43-57
31. O'Neill, M. E., and Mathews, K. L. (2002) Levene tests of homogeneity of variance for general block and treatment designs. Biometrics 58, 216-224
32. Wilcoxon, F. (1946) Individual comparisons of grouped data by ranking methods. Journal of economic entomology 39, 269
33. Madar, V., and Batista, S. (2016) FastLSU: a more practical approach for the Benjamini-Hochberg FDR controlling procedure for huge-scale testing problems. Bioinformatics 32, 1716-1723
34. Revelle, W. (1979) Hierarchical Cluster Analysis And The Internal Structure Of Tests. Multivariate behavioral research 14, 57-74
35. Zhou, G., and Xia, J. (2018) OmicsNet: a web-based tool for creation and visual analysis of biological networks in 3D space. Nucleic acids research 46, W514-W522
36. Lagorce-Pages, C., Paraf, F., Wendum, D., Martin, A., and Flejou, J. F. (2004) Expression of inflammatory secretory phospholipase A2 and cytosolic phospholipase A2 in premalignant and malignant Barrett's esophagus. Virchows Archiv: an international journal of pathology 444, 426-435
37. Zhang, C., Yu, H., Xu, H., and Yang, L. (2015) Expression of secreted phospholipase A2-Group IIA correlates with prognosis of gastric adenocarcinoma. Oncology letters 10, 3050-3058
38. Yoo, Y. S., Lim, S. C., and Kim, K. J. (2011) Prognostic significance of cytosolic phospholipase A2 expression in patients with colorectal cancer. J Korean Surg Soc 80, 397-403
39. Zhu, X., Wang, K., Liu, G., Wang, Y., Xu, J., Liu, L., Li, M., Shi, J., Aa, J., and Yu, L. (2017) Metabolic Perturbation and Potential Markers in Patients with Esophageal Cancer. Gastroenterol Res Pract 2017, 5469597
40. Nowotarski, S. L., Woster, P. M., and Casero, R. A., Jr. (2013) Polyamines and cancer: implications for chemotherapy and chemoprevention. Expert Rev Mol Med 15, e3
41. Pavlides, S., Tsirigos, A., Migneco, G., Whitaker-Menezes, D., Chiavarina, B., Flomenberg, N., Frank, P. G., Casimiro, M. C., Wang, C., Pestell, R. G., Martinez-Outschoorn, U. E., Howell, A., Sotgia, F., and Lisanti, M. P. (2010) The autophagic tumor stroma model of cancer: Role of oxidative stress and ketone production in fueling tumor cell metabolism. Cell Cycle 9, 3485-3505
42. Martinez, Y., Li, X., Liu, G., Bin, P., Yan, W., Mas, D., Valdivie, M., Hu, C. A., Ren, W., and Yin, Y. (2017) The role of methionine on metabolism, oxidative stress, and diseases. Amino acids 49, 2091-2098
43. Hardbower, D. M., de Sablet, T., Chaturvedi, R., and Wilson, K. T. (2013) Chronic inflammation and oxidative stress: the smoking gun for Helicobacter pylori-induced gastric cancer? Gut Microbes 4, 475-481
44. Reuter, S., Gupta, S. C., Chaturvedi, M. M., and Aggarwal, B. B. (2010) Oxidative stress, inflammation, and cancer: how are they linked? Free Radic Biol Med 49, 1603-1616
45. Zeng, R., Duan, L., Kong, Y., Liang, Y., Wu, X., Wei, X., and Yang, K. (2013) Clinicopathological and prognostic role of MMP-9 in esophageal squamous cell carcinoma: a meta-analysis. Chin J Cancer Res 25, 637-645
46. Zhang, B. G., Du, T., Zang, M. D., Chang, Q., Fan, Z. Y., Li, J. F., Yu, B. Q., Su, L. P., Li, C., Yan, C., Gu, Q. L., Zhu, Z. G., Yan, M., and Liu, B. (2014) Androgen receptor promotes gastric cancer cell migration and invasion via AKT-phosphorylation dependent upregulation of matrix metalloproteinase 9. Oncotarget 5, 10584-10595
47. Yang, L., Ren, Y., Yu, X., Qian, F., Bian, B. S., Xiao, H. L., Wang, W. G., Xu, S. L., Yang, J., Cui, W., Liu, Q., Wang, Z., Guo, W., Xiong, G., Yang, K., Qian, C., Zhang, X., Zhang, P., Cui, Y. H., and Bian, X. W. (2014) ALDH1A1 defines invasive cancer stem-like cells and predicts poor prognosis in patients with esophageal squamous cell carcinoma. Modern pathology: an official journal of the United States and Canadian Academy of Pathology, Inc 27, 775-783

1. A method of detecting esophageal adenocarcinoma (EAC) or of monitoring EAC disease progression in a subject, the method comprising:
obtaining a biological sample from the subject;
measuring with a quantitative analytical method at least one metabolite selected from citrulline, C0, SM OH C16:1, glutamic acid, creatinine, histidine, glycine, phenylalanine, methionine, glutamine, serine, tyrosine, alanine, isoleucine, SM C24:0, C2, SM C16:0, taurine, valine, SM OH C22:1, leucine, sarcosine, SM C24:1, threonine, asparagine, lysine, Met-SO, SM C18:0, proline, t4-OH-Pro, C3-DC C4-OH, SM C26:0, aspartic acid, C14:2-OH, histamine, Met-SO/Met, asymmetric dimethyl arginine (ADMA), arginine, a glycerophospholipid, a sphingolipid and combinations thereof;
determining a metabolomic biosignature of EAC disease progression based on a comparison of quantitative data for the at least one metabolite to corresponding data obtained for at least one reference sample; and
detecting EAC or active EAC disease progression in the subject if:
(i) the quantity of the at least one metabolite in the sample from the subject is greater than that found in the at least one reference sample, wherein the at least one metabolite is selected from the group consisting of: citrulline, C0, SM OH C16:1, glutamic acid, creatinine, histidine, glycine, phenylalanine, methionine, glutamine, serine, tyrosine, alanine, isoleucine, SM C24:0, C2, SM C16:0, taurine, valine, SM OH C22:1, leucine, sarcosine, SM C24:1, threonine, asparagine, lysine, Met-SO, SM C18:0, proline, t4-OH-Pro, C3-DC C4-OH, SM C26:0, aspartic acid, C14:2-OH, histamine, Met-SO/Met, asymmetric dimethyl arginine (ADMA) and combinations thereof, or
(ii) the quantity of the at least one metabolite in the sample from the subject is less than that found in the at least one reference sample, wherein the at least one metabolite is selected from arginine, proline, serine, glutamine, histidine, a glycerophospholipid, a sphingolipid and combinations thereof.

2. (canceled)

3. The method according to claim 1, wherein the at least one metabolite comprises a panel of metabolites selected from the group consisting of:
a) a panel comprising taurine, methionine, methionine sulfoxide (Met-SO), and trans-4-hydroxyproline (t4-OH-Pro);
b) a panel comprising glutamic acid, glycine, histidine, phenylalanine, methionine, serine, glutamine, tyrosine, alanine, isoleucine, valine, leucine, threonine, asparagine, lysine, proline, and aspartic acid; and
c) a panel comprising citrulline, C0, SM OH C16:1, glutamic acid, creatinine, histidine, glycine, phenylalanine, methionine, glutamine, serine, tyrosine, alanine, isoleucine, SM C24:0, C2, SM C16:0, taurine, valine, SM OH C22:1, leucine, sarcosine, SM C24:1, threonine, asparagine, lysine, Met-SO, SM C18:0, proline, t4-OH-Pro, C3-DC C4-OH, SM C26:0, aspartic acid, C14:2-OH, and histamine.

4. The method according to claim 3, wherein the panel of metabolites comprises glutamic acid, glycine, histidine, phenylalanine, methionine, serine, glutamine, tyrosine, alanine, isoleucine, valine, leucine, threonine, asparagine, lysine, proline, and aspartic acid.

5. The method according to claim 1, wherein the quantity of the at least one metabolite in the sample from the subject is at least 2.0 times greater than that found in the at least one reference sample, wherein the at least one metabolite is selected from citrulline, C0, SM OH C16:1, glutamic acid, creatinine, histidine, glycine, phenylalanine, methionine, glutamine, serine, tyrosine, alanine, isoleucine, SM C24:0, C2, SM C16:0, taurine, valine, SM OH C22:1, leucine, sarcosine, SM C24:1, threonine, asparagine, lysine, Met-SO, SM C18:0, proline, t4-OH-Pro, C3-DC C4-OH, SM C26:0, aspartic acid, C14:2-OH, histamine, and combinations thereof.

6. The method according claim 1, wherein the quantitative analytical method comprises mass spectrometry.

7. The method according to claim 6, wherein the mass spectrometry comprises liquid chromatography-tandem mass spectrometry (LC-MS/MS).

8. The method according to claim 7, wherein the LC-MS/MS further comprises analyzing in multiple reaction monitoring (MRM) in positive mode of electrospray ionization (ESI).

9. The method according to claim 1, wherein the at least one reference sample is obtained from at least one reference subject with metaplasia.

10. The method according to claim 1, wherein the biological sample from the subject and the at least one reference sample are gastric fluid samples.

11. The method according to claim 1, wherein active EAC disease progression is progression of the disease from metaplasia to dysplasia and/or dysplasia to EAC.

12-13. (canceled)

14. The method according to claim 1, wherein the quantity of the at least one metabolite in the sample from the subject is at least 1.5 times greater than that found in the at least one reference sample, wherein the at least one metabolite is selected from Met-SO, Met-SO/Met, asymmetric dimethyl arginine (ADMA), t4-OH-Pro, alanine, isoleucine, tyrosine, and combinations thereof.

15-23. (canceled)

24. The method according to claim 1, wherein the glycerophospholipid is lysoPC a C26:0, PC aa C28:1, PC aa C34:2, PC aa C36:1, PC aa C36:2, PC aa C40:4, PC aa C42:1, PC ae C36:2, PC ae C38:1, PC ae C42:2, PC ae C42:3, or a combination thereof.

25. The method according to claim 1, wherein the sphingolipid is SM C20:2, SM OH C16:1, SM OH C22:1, or a combination thereof.

26. The method according to claim 1, wherein the quantity of the at least one metabolite in the sample from the subject is about 1.5 times less than that found in the at least one reference sample, wherein the at least one metabolite is selected from arginine, proline, serine, glutamine, histidine, a glycerophospholipid, a sphingolipid, and combinations thereof.

27-33. (canceled)

34. A method of monitoring esophageal adenocarcinoma (EAC) disease progression in a subject, the method comprising:
obtaining a biological sample from the subject;
measuring with a quantitative analytical method at least one metabolite selected from citrulline, C0, SM OH C16:1, glutamic acid, creatinine, histidine, glycine, phenylalanine, methionine, glutamine, serine, tyrosine, alanine, isoleucine, SM C24:0, C2, SM C16:0, taurine, valine, SM OH C22:1, leucine, sarcosine, SM C24:1, threonine, asparagine, lysine, Met-SO, SM C18:0, proline, t4-OH-Pro, C3-DC C4-OH, SM C26:0, aspartic acid, C14:2-OH, histamine, Met-SO/Met, asymmetric dimethyl arginine (ADMA), arginine, a glycerophospholipid, a sphingolipid and combinations thereof;
determining a metabolomic biosignature of EAC disease progression based on a comparison of quantitative data for the at least one metabolite to corresponding data obtained for at least one reference sample; and
detecting active EAC disease progression in the subject if the quantity of the at least one metabolite in the sample from the subject is greater than that found in the at least one reference sample.

35. The method of claim 34, wherein the at least one metabolite is selected from the group consisting of: citrulline, C0, SM OH C16:1, glutamic acid, creatinine, histidine, glycine, phenylalanine, methionine, glutamine, serine, tyrosine, alanine, isoleucine, SM C24:0, C2, SM C16:0, taurine, valine, SM OH C22:1, leucine, sarcosine, SM C24:1, threonine, asparagine, lysine, Met-SO, SM C18:0, proline, t4-OH-Pro, C3-DC C4-OH, SM C26:0, aspartic acid, C14:2-OH, histamine, Met-SO/Met, asymmetric dimethyl arginine (ADMA) and combinations thereof.

36. The method of claim 34, wherein the quantity of at least one metabolite in the sample from the subject selected from the group consisting of: arginine, proline, serine, glutamine, histidine, a glycerophospholipid, a sphingolipid and combinations thereof is less than that found in the at least one reference sample.
